(19) United States
(12) Patent Application Publication
(10) Pub. No.: US 2025/0336526 A1
(43) Pub. Date: Oct. 30, 2025

(54) DIAGNOSIS ASSISTANCE DEVICE, LEARNING MODEL CREATION DEVICE, DIAGNOSIS ASSISTANCE METHOD, LEARNING MODEL CREATION METHOD, AND PROGRAM

(71) Applicant: NIHON UNIVERSITY, Tokyo (JP)

(72) Inventor: Yasuyuki SUZUKI, Tokyo (JP)

(21) Appl. No.: 19/183,563

(22) Filed: Apr. 18, 2025

Related U.S. Application Data
(63) Continuation-in-part of application No. PCT/JP2023/038192, filed on Oct. 23, 2023.

(30) Foreign Application Priority Data
Oct. 28, 2022 (JP) 2022-173465

(51) Int. Cl. G16H 50/20; A61B 6/50; G16H 20/10; G16H 20/40

(52) U.S. Cl. CPC G16H 50/20; A61B 6/503; A61B 6/507; G16H 20/10; G16H 20/40

(57) ABSTRACT

A diagnosis assistance device receives an automatically quantified value of myocardial ischemia, and cardiac function information and phase information acquired by performing stress myocardial scintigraphy and acquires and outputs at least one of a reperfusion therapy prediction result, a heart failure onset prediction result, a cardiac death prediction result, an all-cause mortality prediction result, and a coronary artery disease prediction result on the basis of the automatically quantified value of the myocardial ischemia, the cardiac function information, the phase information, and a trained model. The trained model is obtained by performing machine learning on a relationship between a combination of the automatically quantified value of the myocardial ischemia, the cardiac function information, and the phase information and at least one of the reperfusion therapy result, the heart failure onset prediction result, the cardiac death prediction result, the all-cause mortality prediction result, and the coronary artery disease prediction result.

### TECHNICAL FIELD

The present invention relates to a diagnosis assistance device, a learning model creation device, a diagnosis assistance method, a learning model creation method, and a program.

Priority is claimed on Japanese Patent Application No. 2022-173465, filed Oct. 28, 2022, the content of which is incorporated herein by reference.

### BACKGROUND ART

Stress myocardial scintigraphy is a test in which a blood flow distribution in the left ventricular myocardium is imaged as one of standard tests targeting ischemic heart diseases such as angina pectoris and myocardial infarction. The stress myocardial scintigraphy is a nuclear medicine test in which a radioisotope preparation having the property of distributing in the myocardium in proportion to the myocardial blood flow is intravenously injected and blood flow distributions in the myocardium during stress and rest are imaged with an imaging device such as a gamma camera.

This test provides physiological myocardial blood flow information (mainly information about an amount of ischemia in an ischemic site), which is an important diagnostic basis for predicting the prognosis of ischemic heart disease, but a semi-quantitative score based on visual determination is generally used for diagnosis and the diagnosis is useful for stratifying event risks including all-cause mortality, cardiac death, the onset of acute coronary syndrome, and implementation of reperfusion therapy and predicting prognosis as reported (see, for example, Non-Patent Document 1). Here, the term “semi-quantitative score” is used to indicate a quantitative score based on visual assessment by the human eye.

As semi-quantitative score calculation methods, a summed stress score (SSS) calculated by visually assessing myocardial blood flow images during stress, a summed rest score (SRS) calculated based on images during rest, and a summed difference score (SDS) expressing a blood flow distribution difference between a rest period and a stress period are widely used in general.

Moreover, as an alternative to visual diagnosis, a diagnostic method in which a plurality of normal myocardial scintigraphy images obtained in the past are accumulated and regions showing a blood flow distribution below an average value of normal images are diagnosed as ischemic regions, i.e., abnormal, using an automatically quantified value of myocardial ischemia called total perfusion deficit (TPD) has also been reported and it has been reported that both TPD and semi-quantitative scores have equivalent diagnostic performance.

Both the semi-quantitative assessment score and the TPD are indices that correlate with the severity of myocardial ischemia and an area of the ischemic region (the abnormal region) in the left ventricular myocardium and are considered to be mutually compatible indices. On the other hand, it is assumed that the detection sensitivity of the TPD for ischemic heart disease is higher than that of the semi-quantitative score in a case where abnormal findings, which are not easily detected by visual assessment, are easily detected by the TPD and the like in medical cases where mild ischemia is widespread throughout the myocardium. Moreover, because the semi-quantitative score (especially a score obtained by an experienced radiologist) is calculated after removing clinically insignificant image noise and a possibility that the semi-quantitative score will have a diagnostic capability with higher specificity than the TPD is considered, a possibility that it will play a complementary role in determining whether an image is normal or abnormal is also considered.

In stress myocardial scintigraphy, video information that enables observation of left ventricular wall motion is usually collected by an electrocardiogram-gated imaging method and it is possible to automatically calculate a left ventricular ejection fraction (EF) indicating left ventricular contractility and a left ventricular end-diastolic volume (EDV) from data thereof (see, for example, Non-Patent Document 2).

It is known that EF and EDV information obtained by stress myocardial scintigraphy is important information for prognostic prediction. A left ventricular volume ratio during stress and rest (a transient ischemic dilation (TID) ratio) is useful for detecting a severe coronary artery lesion and a multivessel coronary artery lesion that are easily overlooked by the semi-quantitative score and TPD mentioned above.

Furthermore, it is possible to measure quantitative indices related to a timing (a phase) of contraction and expansion of each site of the left ventricle from video information obtained by the electrocardiogram-gated imaging method. The repeated movement of expansion and contraction of the myocardium in which a heart rate is in a steady state is considered to be a periodic function, and it is observed as an increase or a decrease in a gamma ray count in each site of the myocardium in actual image data (a phase of an increase or a decrease in a gamma ray count value for the contraction and expansion of the heart in each site of the myocardium). Phase information can be expressed by a histogram with a phase at which the gamma ray count reaches its peak on the horizontal axis (the scale is usually expressed in analogy with trigonometric functions, ranging from 0° to 360°) and the number of segments for the number of voxels) at which the count peaks at that phase on the vertical axis.

From this histogram information, it is possible to calculate a bandwidth (BW), indicating the horizontal spread of a histogram (a maximum value of a phase shift), a standard deviation (SD) indicating a statistical variation, and an entropy, which represents the disorder of the phase information. It has been reported that these information items also have additional prognostic predictive values with respect to the semi-quantitative score, TPD, EF, and EDV mentioned above.

The following techniques are known for predicting a possibility of the onset of future disease. For example, a technique using dynamic analysis of biophysical signals is known (see, for example, Patent Document 1). This technique, for example, characterizes and identifies nonlinear dynamic properties of biophysical signals, such as photoplethysmography signals and/or cardiac signals, and facilitates one or more dynamic analyses that can predict the presence and/or localization of a disease or pathological condition, or an index thereof.

Moreover, a technique using an artificial intelligence algorithm is known (see, for example, Patent Document 2). This technique includes the steps of acquiring input data based on a subject's medical diagnosis data, generating output data indicating a year-specific disease onset possibility from the input data using a trained artificial intelligence model, determining at least one item having a relatively high contribution degree with respect to a result of the output data, and outputting information about the year-specific disease onset possibility and at least one item.

### CITATION LIST

### Patent Document

Patent Document 1: Published Japanese Translation No. 2022-538988 of the PCT International Publication
Patent Document 2: Published Japanese Translation No. 2022-551005 of the PCT International Publication

### Non-Patent Document

[Non-Patent Document 1] Shunichi Yoda, Kanae Nakanishi, Ayako Tano, Yusuke Hori, Yasuyoki Suzuki, Naoya Matsumoto, Atsushi Hirayama, “Usefulness of automated assessment of nuclear cardiology for prediction of major cardiac events in Japanese patients with known or suspected coronary artery disease: Comparison with conventional visual assessment in a large-scale prognostic study,” Journal of Cardiology 64 (2014), 395-400
[Non-Patent Document 2] Kenichi Nakajima. Shinro Matsuo, Chio Okuyama, Tsuguru Hatta, Kazumasa Tsukamoto, Shigeyuki Nishimura, Akira Yamashina, Hideo Kusuoka, Tsunchiko Nishimura, “Cardiac event risk in Japanese subjects estimated using gated myocardial perfusion imaging, in conjunction with diabetes mellitus and chronic kidney disease,” Circulation Journal Vol, 76, January 2012

### SUMMARY OF INVENTION

### Technical Problem

Semi-quantitative scoring using an SSS and an SRS is a technique that requires a certain level of skill. It has been reported that cardiac event prediction diagnosis functions using an SSS and a TPD are statistically equivalent. However, it has been reported that false negatives can occur, albeit rarely, in all diagnostic indices. The current challenge is to reduce a false negative rate while maintaining the sensitivity and specificity of the test.

As a means for reducing false negatives, there is a method for decreasing cutoff values of the SSS and the TPD for diagnosis. However, there is a concern that this will increase false positives and lead to an increase in the number of unnecessary additional tests. Moreover, a blood flow defect region expressed by the TPD may represent a region that cannot be visually detected as a defect region and it is considered that they are not completely equivalent indices. However, when there is a discrepancy between the assessments according to the semi-quantitative score and the TPD, there is no clear criterion for determining an item to be used preferentially.

As a method for complementing false negatives from the semi-quantitative scoring and TPD, a method in which cardiac function information (EF) and clinical information (age, the presence or absence of diabetes, and an index of a renal function) are added to the semi-quantitative score obtained by visual assessment from stress myocardial scintigraphy images to predict the risk of cardiac events has been reported. There is also a method in which prognostic information is reflected in an image reading system on the basis of results of past large-scale clinical studies including the above information.

There is a characteristic that an abnormal finding of a blood flow distribution represented by myocardial scintigraphy tends to be visually recognized as a blood flow defect in a region with the poorest blood flow distribution in the entire myocardium, whereas it is difficult to visually recognize the blood flow defect when there is multiple coronary artery stenosis and the blood flow of the myocardium deteriorates overall and a balanced ischemia state is likely to occur. This is said to be a weakness of myocardial scintigraphy in diagnosing ischemic heart disease. There are mainly three coronary arteries, which are blood vessels that nourish the heart. When a coronary artery lesion is found in only one, it is referred to as a “single-vessel lesion.” When a stenosis lesion (a coronary artery lesion) is found in two or more coronary arteries, it is referred to as a “multivessel lesion.”

Although there is also a system using supervised learning with an artificial neural network to learn from image interpretation by an expert and diagnose blood flow abnormalities in stress myocardial scintigraphy, its use is limited to identifying abnormal sites on an image (regions of ischemia/myocardial infarction).

As a means for use in event risk stratification other than imaging test information, a Suita score of a clinical risk score model for predicting the risk of developing a cardiac event within 10 years by scoring age, sex, blood pressure, LDL cholesterol level, HDL cholesterol level, the presence or absence of impaired glucose tolerance, a smoking history, and a family history of premature coronary artery disease is known.

In Japan, it has been reported that the Suita score reduces risk overestimation compared to a Framingham risk score, a clinical risk score for Westerners. The Suita score allows for stratification of cardiac event risk in urban Japanese residents, but it is generally necessary to confirm the risk assessment by additional imaging tests and the like in cases where the risk assessment is moderate or higher.

As a simple test method for identifying the risk of ischemic heart disease, there is a coronary artery calcium scan that can quantitatively detect calcium (calcified sites) in blood vessels accumulated due to coronary arteriosclerosis. The coronary artery calcium scan does not use a contrast agent, and identifies regions showing CT values corresponding to calcified sites in the coronary artery from cardiac computed tomography (CT) images captured by electrocardiogram-gated imaging, and calculates a coronary artery calcium score (CACS) as a quantitative value of calcification in the coronary artery. It has been reported that the CACS contributes to the stratification of cardiac event risk in combination with exercise stress electrocardiogram testing and stress myocardial scintigraphy.

An objective of the present invention is to provide a diagnosis assistance device, a learning model creation device, a diagnosis assistance method, a learning model creation method, and a program for enabling the acquisition of at least one of a predicted value of a result of performing reperfusion therapy, a result of predicting the onset of heart failure, a result of predicting cardiac death, a result of predicting all-cause mortality, and a result of predicting coronary artery disease for a subject.

### Solution to Problem

(1) According to an aspect of the present invention, there is provided a diagnosis assistance device including: a reception unit configured to receive an automatically quantified value of myocardial ischemia of a subject and cardiac function information and phase information acquired when stress myocardial scintigraphy is performed on the subject; a processing unit including a trained model for acquiring at least one of a predicted value of a result of performing reperfusion therapy, a result of predicting the onset of heart failure, a result of predicting cardiac death, a result of predicting all-cause mortality, and a result of predicting coronary artery disease on the basis of the automatically quantified value of the myocardial ischemia, the cardiac function information, and the phase information received by the reception unit and a trained model; and an output unit configured to output at least one of the predicted value of the result of performing the reperfusion therapy, the result of predicting the onset of the heart failure, the result of predicting the cardiac death, the result of predicting the all-cause mortality, and the result of predicting the coronary artery disease acquired by the processing unit, wherein the phase information is obtained by acquiring a phase of an increase or a decrease in a gamma ray count for contraction and expansion of a heart in each part of myocardium in a region of interest on an image associated with the contraction and expansion of the heart according to video information of the stress myocardial scintigraphy, and wherein the trained model is obtained by performing machine learning on a relationship between a combination of the automatically quantified value of the myocardial ischemia, the cardiac function information, and the phase information and at least one of the predicted value of the result of performing the reperfusion therapy, the result of predicting the onset of the heart failure, the result of predicting the cardiac death, the result of predicting the all-cause mortality, and the result of predicting the coronary artery disease.
(2) In the diagnosis assistance device according to the aspect of the present invention, the reception unit receives a visual semi-quantitative index of the subject and the cardiac function information and the phase information acquired when the stress myocardial scintigraphy is performed on the subject, the processing unit acquires at least one of the predicted value of the result of performing the reperfusion therapy, the result of predicting the onset of the heart failure, the result of predicting the cardiac death, the result of predicting the all-cause mortality, and the result of predicting the coronary artery disease on the basis of the visual semi-quantitative index, the cardiac function information, and the phase information received by the reception unit, and the trained model is obtained by performing machine learning on a relationship between a combination of the visual semi-quantitative index, the cardiac function information, and the phase information and at least one of the predicted value of the result of performing the reperfusion therapy, the result of predicting the onset of the heart failure, the result of predicting the cardiac death, the result of predicting the all-cause mortality, and the result, of predicting the coronary artery disease.
(3) In the diagnosis assistance device according to the aspect of the present invention, the reception unit receives at least one of an index for predicting the onset of coronary artery disease acquired before the stress myocardial scintigraphy is performed on the subject, a coronary artery calcium score of the subject acquired when the stress myocardial scintigraphy is performed, a body mass index of the subject, and a left ventricular volume ratio during stress and rest of the subject, the processing unit acquires at least one of the predicted value of the result of performing the reperfusion therapy, the result of predicting the onset of the heart failure, the result of predicting the cardiac death, the result of predicting the all-cause mortality, and the result of predicting the coronary artery disease on the basis of at least one of the index for predicting the onset of the coronary artery disease, the coronary artery calcium score, the body mass index, and the left ventricular volume ratio received by the reception unit, the automatically quantified value of the myocardial ischemia, the cardiac function information, the phase information, and the trained model, and the trained model is obtained by performing machine learning on a relationship between a combination of at least one of the index for predicting the onset of the coronary artery disease, the coronary artery calcium score, the body mass index, and the left ventricular volume ratio, the automatically quantified value of the myocardial ischemia, the cardiac function information, and the phase information and at least one of the predicted value of the result of performing the reperfusion therapy, the result of predicting the onset of the heart failure, the result of predicting the cardiac death, the result of predicting the all-cause mortality, and the result of predicting the coronary artery disease.
(4) In the diagnosis assistance device according to the aspect of the present invention, the cardiac function information includes a left ventricular ejection fraction.
(5) In the diagnosis assistance device according to the aspect of the present invention, the phase information includes at least one of a standard deviation, a phase bandwidth, and an entropy obtained from measurement of timings of contraction and expansion of the myocardium.
(6) According to an aspect of the present invention, there is provided a learning model creation device including: a reception unit configured to receive a learning dataset in which an automatically quantified value of myocardial ischemia of a subject and cardiac function information and phase information acquired when stress myocardial scintigraphy is performed on the subject is included as learning data and at least one of a predicted value of a result of performing reperfusion therapy on the subject, a result of the onset of heart failure, a result of cardiac death, a result of all-cause mortality, and a result of coronary artery disease is included as training data; a processing unit configured to create a learning model by performing machine learning on a relationship of the automatically quantified value of the myocardial ischemia, the cardiac function information, the phase information, and at least one of the predicted value of the result of performing the reperfusion therapy, the result of the onset of the heart failure, the result of the cardiac death, the result of the all-cause mortality, and the result of the coronary artery disease using the automatically quantified value of the myocardial ischemia, the cardiac function information, and the phase information as explanatory variables and using at least one of the predicted value of the result of performing the reperfusion therapy, the result of the onset of the heart failure, the result of the cardiac death, the result of the all-cause mortality, and the result of the coronary artery disease ax an objective variable on the basis of the learning dataset received by the reception unit; and an output unit configured to output the learning model created by the processing unit, wherein the phase information is obtained by acquiring a phase of an increase or a decrease in a gamma ray count in each part of myocardium in a region of interest on an image associated with contraction and expansion of a heart according to video information of the stress myocardial scintigraphy.
(7) In the learning model creation device according to the aspect of the present invention, the reception unit receives a learning dataset in which a visual semi-quantitative index of the subject and the cardiac function information and the phase information acquired when the stress myocardial scintigraphy is performed on the subject is included as learning data and at least one of the predicted value of the result of performing the reperfusion therapy on the subject, the result of the onset of the heart failure, the result of the cardiac death, the result of the all-cause mortality, and the result of the coronary artery disease is included as training data, and the processing unit configured to create a learning model by performing machine learning on a relationship of the visual semi-quantitative index, the cardiac function information, the phase information, and at least one of the predicted value of the result of performing the reperfusion therapy, the result of the onset of the heart failure, the result of the cardiac death, the result of the all-cause mortality, and the result of the coronary artery disease using the visual semi-quantitative index, the cardiac function information, and the phase information as explanatory variables and using at least one of the predicted value of the result of performing the reperfusion therapy, the result of the onset of the heart failure, the result of the cardiac death, the result of the all-cause mortality, and the result of the coronary artery disease as an objective variable on the basis of the learning dataset received by the reception unit.
(8) In the learning model creation device according to the aspect of the present invention, the reception unit receives a learning dataset in which at least one of an index for predicting the onset of coronary artery disease acquired before the stress myocardial scintigraphy is performed on the subject, a coronary artery calcium score of the subject acquired when the stress myocardial scintigraphy is performed, a body mass index of the subject, and a left ventricular volume ratio during stress and rest of the subject is further included as learning data, and the processing unit creates a learning model by performing machine learning on a relationship of a visual semi-quantitative index, the cardiac function information, the phase information, and at least one of the predicted value of the result of performing the reperfusion therapy, the result of the onset of the heart failure, the result of the cardiac death, the result of the all-cause mortality, and the result of the coronary artery disease using at least one of the index for predicting the onset of the coronary artery disease, the coronary artery calcium score, the body mass index, and the left ventricular volume ratio, the automatically quantified value of the myocardial ischemia, the cardiac function information, and the phase information as explanatory variables and using at least one of the predicted value of the result of performing the reperfusion therapy, the result of the onset of the heart failure, the result of the cardiac death, the result of the all-cause mortality, and the result of the coronary artery disease as an objective variable on the basis of the learning dataset received by the reception unit.
(9) According to an aspect of the present invention, there is a provided a diagnosis assistance method to be executed by a computer, the diagnosis assistance method including: a step of receiving an automatically quantified value of myocardial ischemia of a subject and cardiac function information and phase information acquired when stress myocardial scintigraphy is performed on the subject; a step of acquiring at least one of a predicted value of a result of performing reperfusion therapy, a result of predicting the onset of heart failure, a result of predicting cardiac death, a result of predicting all-cause mortality, and a result of predicting coronary artery disease on the basis of the automatically quantified value of the myocardial ischemia, the cardiac function information, and the phase information received in the receiving step and a trained model; and a step of outputting at least one of the predicted value of the result of performing the reperfusion therapy, the result of predicting the onset of the heart failure, the result of predicting the cardiac death, the result of predicting the all-cause mortality, and the result of predicting the coronary artery disease that have been acquired, wherein the phase information is obtained by acquiring a phase of an increase or a decrease in a gamma ray count in each part of myocardium in a region of interest on an image associated with contraction and expansion of a heart according to video information of the stress myocardial scintigraphy, and wherein the trained model is obtained by performing machine learning on a relationship between a combination of the automatically quantified value of the myocardial ischemia, the cardiac function information, and the phase information and at least one of the predicted value of the result of performing the reperfusion therapy, the result of predicting the onset of the heart failure, the result of predicting the cardiac death, the result of predicting the all-cause mortality, and the result of predicting the coronary artery disease.
(10) According to an aspect of the present invention, there is a provided a learning model creation method to be executed by a computer, the learning model creation method including: a step of receiving a learning dataset in which an automatically quantified value of myocardial ischemia of a subject and cardiac function information and phase information acquired when stress myocardial scintigraphy is performed on the subject is included as learning data and at least one of a predicted value of a result of performing reperfusion therapy on the subject, a result of the onset of heart failure, a result of cardiac death, a result of all-cause mortality, and a result of coronary artery disease is included as training data; a step of creating a learning model by performing machine learning on a relationship of the automatically quantified value of the myocardial ischemia, the cardiac function information, the phase information, and at least one of the predicted value of the result of performing the reperfusion therapy, the result of the onset of the heart failure, the result of the cardiac death, the result of the all-cause mortality, and the result of the coronary artery disease using the automatically quantified value of the myocardial ischemia, the cardiac function information, and the phase information as explanatory variables and using at least one of the predicted value of the result of performing the reperfusion therapy, the result of the onset of the heart failure, the result of the cardiac death, the result of the all-cause mortality, and the result, of the coronary artery disease as an objective variable on the basis of the learning dataset received in the receiving step; and a step of outputting the learning model created in the creating step, wherein the phase information is obtained by acquiring a phase of an increase or a decrease in a gamma ray count in each part of myocardium in a region of interest on an image associated with contraction and expansion of a heart according to video information of the stress myocardial scintigraphy.
(11) According to an aspect of the present invention, there is a provided a program for causing a computer to execute: a step of receiving an automatically quantified value of myocardial ischemia of a subject and cardiac function information and phase information acquired when stress myocardial scintigraphy is performed on the subject; a step of acquiring at least one of a predicted value of a result of performing reperfusion therapy, a result of predicting the onset of heart failure, a result of predicting cardiac death, a result of predicting all-cause mortality, and a result of predicting coronary artery disease on the basis of the automatically quantified value of the myocardial ischemia, the cardiac function information, and the phase information received in the receiving step and a trained model; and a step of outputting at least one of the predicted value of the result of performing the reperfusion therapy, the result of predicting the onset of the heart failure, the result of predicting the cardiac death, the result of predicting the all-cause mortality, and the result of predicting the coronary artery disease that have been acquired, wherein the phase information is obtained by acquiring a phase of an increase or a decrease in a gamma ray count in each part of myocardium in a region of interest on an image associated with contraction and expansion of a heart according to video information of the stress myocardial scintigraphy, and wherein the trained model is obtained by performing machine learning on a relationship between a combination of the automatically quantified value of the myocardial ischemia, the cardiac function information, and the phase information and at least one of the predicted value of the result of performing the reperfusion therapy, the result of predicting the onset of the heart failure, the result of predicting the cardiac death, the result of predicting the all-cause mortality, and the result of predicting the coronary artery disease.
(12) According to an aspect of the present invention, there is a provided a program for causing a computer to execute: a step of receiving a learning dataset in which an automatically quantified value of myocardial ischemia of a subject and cardiac function information and phase information acquired when stress myocardial scintigraphy is performed on the subject is included as learning data and at least one of a predicted value of a result of performing reperfusion therapy on the subject, a result of the onset of heart failure, a result of cardiac death, a result of all-cause mortality, and a result of coronary artery disease is included as training data; a step of creating a learning model by performing machine learning on a relationship of the automatically quantified value of the myocardial ischemia, the cardiac function information, the phase information, and at least one of the predicted value of the result of performing the reperfusion therapy, the result of the onset of the heart failure, the result of the cardiac death, the result of the all-cause mortality, and the result of the coronary artery disease using the automatically quantified value of the myocardial ischemia, the cardiac function information, and the phase information as explanatory variables and using at least one of the predicted value of the result of performing the reperfusion therapy, the result of the onset of the heart failure, the result of the cardiac death, the result of the all-cause mortality, and the result of the coronary artery disease as an objective variable on the basis of the learning dataset received in the receiving step; and a step of outputting the learning model created in the creating step, wherein the phase information is obtained by acquiring a phase of an increase or a decrease in a gamma ray count in each part of myocardium in a region of interest on an image associated with contraction and expansion of a heart according to video information of the stress myocardial scintigraphy.

### Advantageous Effects of Invention

According to the present invention, it is possible to provide a diagnosis assistance device, a learning model creation device, a diagnosis assistance method, a learning model creation method, and a program for enabling the acquisition of at least one of a predicted value of a result of performing reperfusion therapy, a result of predicting the onset of heart failure, a result of predicting cardiac death, a result of predicting all-cause mortality, and a result of predicting coronary artery disease for a subject.

### BRIEF DESCRIPTION OF DRAWINGS

FIG. 1 A diagram showing an example of a diagnosis assistance device according to the present embodiment.

FIG. 2 An explanatory diagram of an automatically quantified value of myocardial ischemia.

FIG. 3A An explanatory diagram of left ventricular contraction phase information.

FIG. 3B A diagram showing a state of the acquisition of the left ventricular contraction phase information.

FIG. 3C An explanatory diagram of the acquisition of the left ventricular contraction phase information.

FIG. 4 A flowchart showing an example of an operation of the diagnosis assistance device according to the embodiment.

FIG. 5 A diagram showing an example of a learning model creation device according to the present embodiment.

FIG. 6 A flowchart showing an example of an operation of the learning model creation device of the present embodiment.

FIG. 7 A diagram showing an example of a diagnosis assistance device according to Modified Example 1 of the embodiment.

FIG. 8 An explanatory diagram of an example of a Suita score.

FIG. 9 A flowchart showing an example of an operation of a diagnosis assistance device according to Modified Example 1 of the embodiment.

FIG. 10 A diagram showing an example of a learning model creation device according to Modified Example 1 of the embodiment.

FIG. 11 A flowchart showing an example of an operation of the learning model creation device according to Modified Example 1 of the embodiment.

FIG. 12 A diagram showing an example of a diagnosis assistance device according to Modified Example 2 of the embodiment,

FIG. 13 An explanatory diagram of a visual semi-quantitative index.

FIG. 14 A flowchart showing an example of an operation of a diagnosis assistance device according to Modified Example 2 of the embodiment.

FIG. 15 A diagram showing an example of a learning model creation device according to Modified Example 2 of the embodiment.

FIG. 16 A flowchart showing an example of an operation of the learning model creation device according to Modified Example 2 of the embodiment.

FIG. 17 A diagram showing an example of a comparison result of receiver operating characteristics of the diagnosis assistance device according to the embodiment.

FIG. 18 A diagram showing the importance of learning data used when a trained model is created according to the embodiment.

FIG. 19 A diagram showing another example of the comparison result of the receiver operating characteristics of the diagnosis assistance device according to the embodiment.

FIG. 20 A diagram showing an example of the diagnostic capability of the diagnosis assistance device according to the embodiment.

FIG. 21 A diagram showing an example of a comparison result of diagnostic capabilities of the diagnosis assistance device according to the embodiment.

FIG. 22 A diagram showing another example of the comparison result of the receiver operating characteristics of the diagnosis assistance device according to the embodiment.

FIG. 23 An example of implementation of explainability.

FIG. 24A A diagram showing an example of a diagnosis result of the diagnosis assistance device of the embodiment.

FIG. 24B Results of comparing the sensitivity, specificity, and accuracy for SDS, sTPD, TIDr, and a case of a diagnosis assistance device 100b of the embodiment.

FIG. 25A A diagram showing an example of a diagnosis result of the diagnosis assistance device of the embodiment.

FIG. 25B Results of comparing the sensitivity, specificity, and accuracy for STPD, dTID, TIDr, and the case of the diagnosis assistance device 100b of the embodiment.

### DESCRIPTION OF EMBODIMENTS

Hereinafter, a diagnosis assistance device, a learning model creation device, a diagnosis assistance method, a learning model creation method, and a program according to embodiments will be described with reference to the drawings. The embodiments to be described below are merely examples and the embodiments to which the present invention is applied are not limited to the following embodiments.

In addition, in all the drawings for describing the embodiments, the same reference signs are used for parts having the same functions and redundant description will be omitted.

Moreover, herein, the term “on the basis of or based on XX” means “on the basis of or based on at least XX” and also includes a case based on another element in addition to XX. Moreover, the term “on the basis of or based on XX” is not limited to a case where XX is directly used and includes a case based on a result of performing a calculation operation or processing on XX. “XX” is any element (e.g., any information).

### EMBODIMENT

### (Diagnosis Assistance Device)

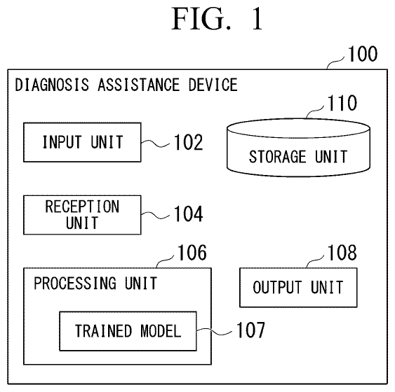

FIG. 1 is a diagram showing an example of a diagnosis assistance device according to the present embodiment. A diagnosis assistance device 100 according to the present embodiment receives subject-related information. The subject-related information includes subject identification information, an automatically quantified value of myocardial ischemia of the subject, and cardiac function information and phase information acquired when stress myocardial scintigraphy is performed.

The cardiac function information includes one or both of a left ventricular ejection fraction (EF) and a left ventricular end-diastolic volume (EDV).

The phase information is left ventricular contraction phase information and includes at least one of a phase bandwidth (BW), a standard deviation, and an entropy.

The diagnosis assistance device 100 acquires at least one of a predicted value of a result of performing reperfusion therapy, a result of predicting the onset of heart failure, a result of predicting cardiac death, a result of predicting all-cause mortality, and a result of predicting coronary artery disease on the basis of the automatically quantified value of the myocardial ischemia, the cardiac function information, and the phase information included in the received subject-related information and a trained model. Here, the trained model is obtained by performing machine learning on a relationship between a combination of the automatically quantified value of the myocardial ischemia, the cardiac function information, and the phase information and at least one of the predicted value of the result of performing the reperfusion therapy, the result of predicting the onset of the heart failure, the result of predicting the cardiac death, the result of predicting the all-cause mortality, and the result of predicting the coronary artery disease.

The diagnosis assistance device 100 outputs the subject identification information and at least one of the predicted value of the result of performing the reperfusion therapy, the result of predicting the onset of the heart failure, the result of predicting the cardiac death, the result of predicting the all-cause mortality, and the result of predicting the coronary artery disease that have been acquired.

The diagnosis assistance device 100 is implemented by a device such as a personal computer, a server, a smartphone, a tablet computer, or an industrial computer. The diagnosis assistance device 100 includes an input unit 102, a reception unit 104, a processing unit 106, an output unit 108, and a storage unit 110.

The input unit 102 inputs information. As an example, the input unit 102 may have an operation unit such as a keyboard or a mouse. In this case, the input unit 102 inputs information according to an operation performed by a user on the operation unit. As another example, the input unit 102 may input information from an external device. The external device may be, for example, a portable storage medium. The subject-related information is input to the input unit 102.

The reception unit 104 acquires the subject-related information from the input unit 102. The reception unit 104 acquires the subject identification information, the automatically quantified value of the myocardial ischemia, the cardiac function information, and the phase information included in the acquired subject-related information, and receives the subject identification information, the automatically quantified value of the myocardial ischemia, the cardiac function information, and the phase information that have been acquired.

The automatically quantified value of the myocardial ischemia is an index that reflects the range and severity of myocardial ischemia.

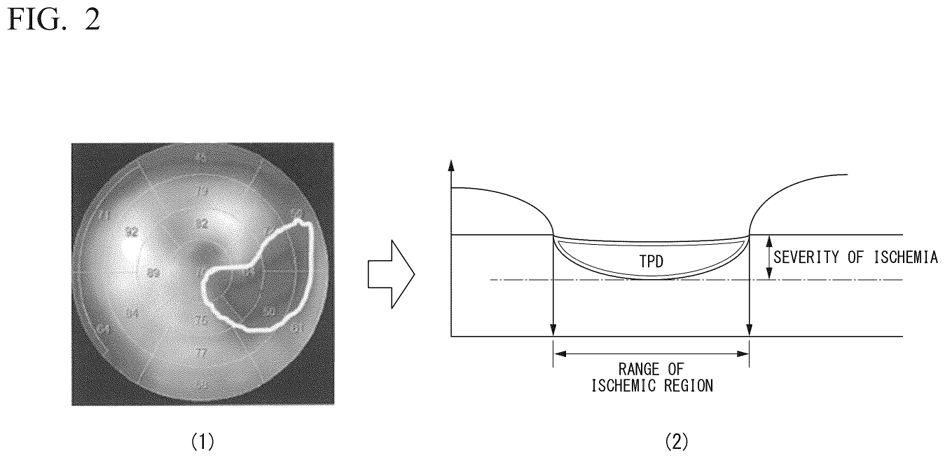

FIG. 2 is an explanatory diagram of the automatically quantified value of the myocardial ischemia. In FIG. 2, (1) is an example of an image of myocardial scintigraphy and (2) is an example of a blood flow distribution profile obtained from (1). The automatically quantified value of the myocardial ischemia is obtained by calculating a blood flow distribution region below a blood flow distribution profile obtained from a normal image of myocardial scintigraphy.

The cardiac function information includes one or both of a left ventricular ejection fraction and a left ventricular end-diastolic volume. The left ventricular ejection fraction and the left ventricular end-diastolic volume can be obtained from video information of the left ventricle obtained by an electrocardiogram-gated imaging method.

Left ventricular contraction phase information includes the phase bandwidth, the standard deviation, and the entropy.

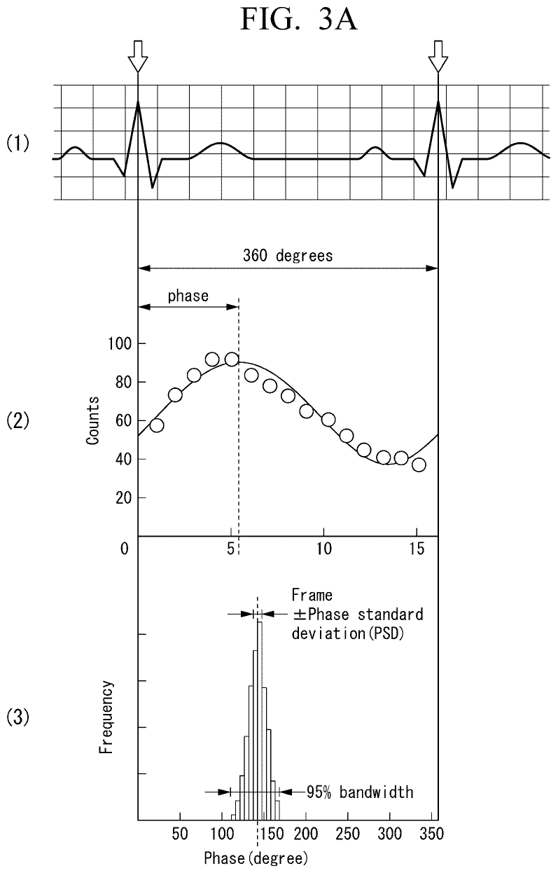

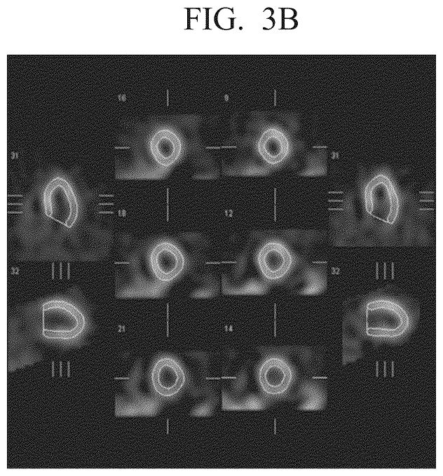

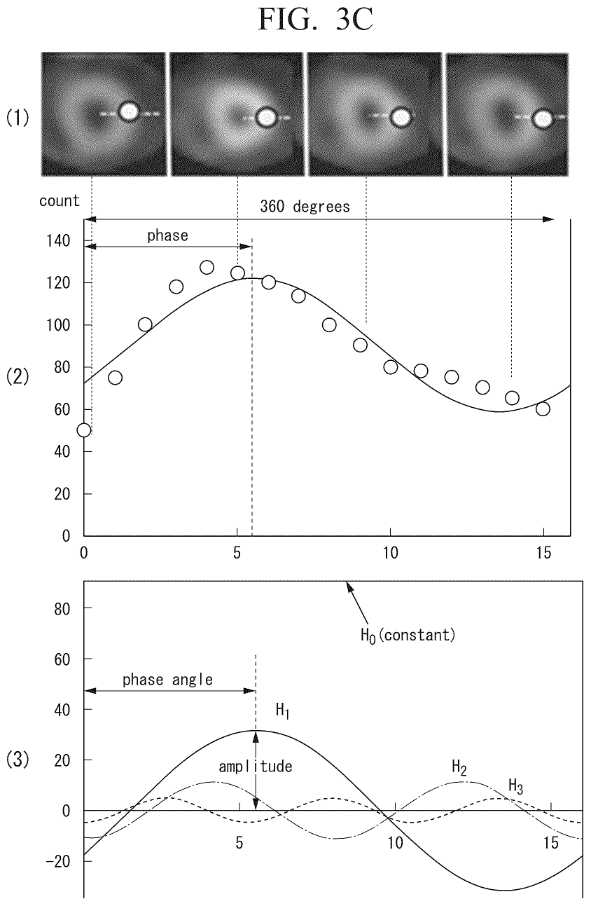

FIG. 3A is an explanatory diagram of left ventricular contraction phase information. In FIG. 3A, (1) is an electrocardiogram, (2) is phase information acquired from all segments of the myocardium, and (3) is a histogram created on the basis of the acquired phase information. The phase bandwidth, standard deviation, and entropy are calculated from the histogram.

The acquisition of the phase information will be described. It is possible to measure quantitative indices related to a timing (a phase) of contraction and expansion of each site of the left ventricle from video information obtained by the electrocardiogram-gated imaging method. The repeated movement of expansion and contraction of the myocardium in which a heart rate is in a steady state is considered to be a periodic function, and it is observed as an increase or a decrease in a gamma ray count in each site of the myocardium in actual image data (a phase of an increase or a decrease in a gamma ray count value for the contraction and expansion of a heart in each site of the myocardium). Phase information can be expressed as a histogram with a phase at which the gamma ray count reaches its peak on the horizontal axis (the scale is usually expressed in analogy with trigonometric functions, ranging from 0° to 360°) and the number of segments (or the number of voxels) at which the count peaks at that phase on the vertical axis.

From this histogram information, it is possible to calculate a bandwidth (BW), indicating the horizontal spread of a histogram (a maximum value of a phase shift), a standard deviation (SD) indicating a statistical variation, and an entropy, which represents the disorder of the phase information.

FIG. 3B is a diagram showing the acquisition of left ventricular contraction phase information. FIG. 3B shows a state of left ventricular contraction. In FIG. 3B, white parts are enhanced in contrast by a contrast agent.

FIG. 3C is an explanatory diagram of the acquisition of left ventricular contraction phase information. A process of acquiring phase information from a left ventricular contraction image will be described with reference to FIG. 3C. In FIG. 3C, (1) shows an example of a left ventricular contraction image, and a region indicated by a white circle is a region of interest. (2) shows a brightness value of the region of interest as an amplitude. (3) shows each phase obtained from an amplitude waveform of a plurality of regions of interest. When the left ventricular contraction is good, the phases are aligned. The description will continue with reference back to FIG. 1.

The processing unit 106 acquires the subject identification information, the automatically quantified value of the myocardial ischemia, the cardiac function information, and the phase information from the reception unit 104. The processing unit 106 includes a trained model 107. The trained model 107 is obtained by performing machine learning on a relationship between a combination of the automatically quantified value of the myocardial ischemia, the cardiac function information, and the phase information and at least one of the result of performing the reperfusion therapy, the result of predicting the onset of the heart failure, the result of predicting the cardiac death, the result of predicting the all-cause mortality, and the result of predicting the coronary artery disease. The processing unit 106 inputs the acquired combination of the automatically quantified value of the myocardial ischemia, the cardiac function information, and the phase information to the trained model 107 and acquires at least one of the result of performing the reperfusion therapy, the result of predicting the onset of the heart failure, the result of predicting the cardiac death, the result of predicting the all-cause mortality, and the result of predicting the coronary artery disease output by the trained model 107 with respect to the input combination of the automatically quantified value of the myocardial ischemia, the cardiac function information, and the phase information.

An example of the reperfusion therapy is a medical procedure for restoring a blood flow in a completely blocked or severely narrowed coronary artery in a heart attack (acute myocardial infarction (MI) or angina pectoris. The reperfusion therapy includes intravascular surgery and surgical procedures using drugs or catheters. The drugs are thrombolytic drugs and fibrinolytic drugs and are used to dissolve blood clots that are blocking or severely narrowing the coronary artery. Intravascular surgery using a catheter is a minimally invasive intravascular procedure referred to as percutaneous coronary intervention (PCI), in which a balloon is expanded in a diseased blood vessel using a catheter and a guide wire to expand the blood vessel, and then a metal tube referred to as a stent is placed to prevent restenosis. Moreover, the reperfusion therapy also includes coronary artery bypass surgery as a surgical procedure.

The onset of the heart failure includes hospitalization for heart failure. The hospitalization for heart failure and cardiac death are included in cardiac events.

The coronary artery disease includes multivessel disease and left main coronary artery disease.

The output unit 108 acquires the subject identification information and at least one of the predicted value of the result of performing the reperfusion therapy, the result of predicting the onset of the heart failure, the result of predicting the cardiac death, the result of predicting the all-cause mortality, and the result of predicting the coronary artery disease from the processing unit 106. The output unit 108 outputs the subject identification information and at least one of the predicted value of the result of performing the reperfusion therapy, the result of predicting the onset of the heart failure, the result of predicting the cardiac death, the result of predicting the all-cause mortality, and the result of predicting the coronary artery disease that have been acquired.

For example, the output unit 108 may output the subject identification information and at least one of the predicted value of the result of performing the reperfusion therapy, the result of predicting the onset of the heart failure, the result of predicting the cardiac death, the result of predicting the all-cause mortality, and the result, of predicting the coronary artery disease by voice or may perform an output process by displaying the subject identification information and at least one of the predicted value of the result of performing the reperfusion therapy, the result of predicting the onset of the heart failure, the result of predicting the cardiac death, the result of predicting the all-cause mortality, and the result of predicting the coronary artery disease on a display unit (not shown).

Moreover, the output unit 108 may associate the subject identification information with at least one of the predicted value of the result of performing the reperfusion therapy, the result of predicting the onset of the heart failure, the result of predicting the cardiac death, the result of predicting the all-cause mortality, and the result of predicting the coronary artery disease and cause the storage unit 110 to store the associated information.

All or some of the input unit 102, the reception unit 104, the processing unit 106, and the output unit. 108 are functional units (hereinafter referred to as software functional units) implemented by a processor such as a central processing unit (CPU) executing a program stored in the storage unit 110.

In addition, all or some of the input unit. 102, the reception unit 104, the processing unit 106, and the output unit 108 may be implemented by hardware such as a large-scale integration (LSI) circuit, an application-specific integrated circuit (ASIC), or a field programmable gate array (FPGA) or may be implemented by a combination of a software function unit and hardware.

### (Operation of Diagnosis Assistance Device 100)

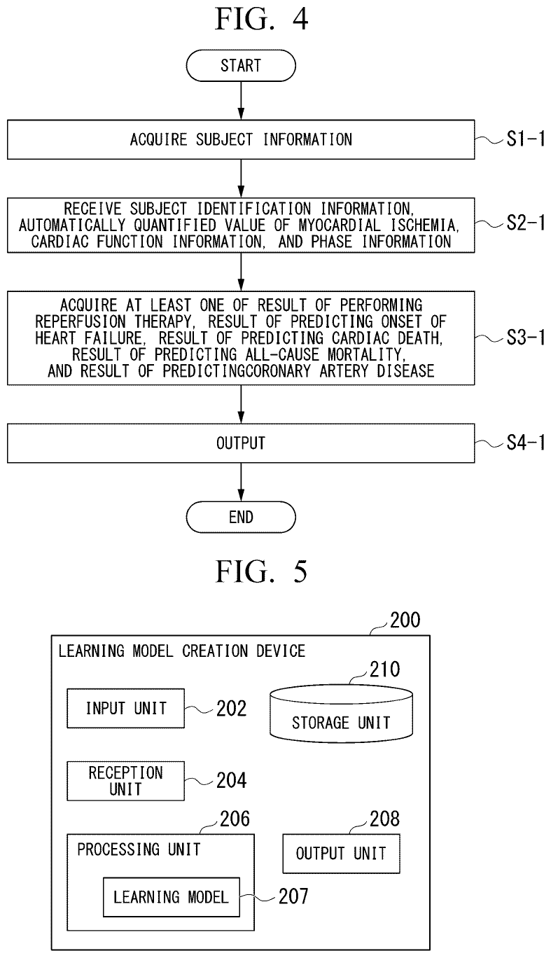

FIG. 4 is a flowchart showing an example of an operation of a diagnosis assistance device according to the embodiment.

### (Step S1-1)

The input unit 102 acquires subject-related information.

### (Step S2-1)

The reception unit 104 acquires the subject-related information from the input unit 102. The reception unit 104 acquires the subject identification information, the automatically quantified value of the myocardial ischemia, the cardiac function information, and the phase information included in the acquired subject-related information, and receives the subject identification information, the automatically quantified value of the myocardial ischemia, the cardiac function information, and the phase information that have been acquired.

### (Step S3-1)

The processing unit 106 acquires the subject identification information, the automatically quantified value of the myocardial ischemia, the cardiac function information, and the phase information from the reception unit 104. The processing unit 106 inputs a combination of the automatically quantified value of the myocardial ischemia, the cardiac function information, and the phase information that have been acquired to the trained model 107a and acquires at least one of the predicted value of the result of performing the reperfusion therapy, the result of predicting the onset of the heart failure, the result of predicting the cardiac death, the result of predicting the all-cause mortality, and the result of predicting the coronary artery disease output by the trained model 107a for the input combination of the automatically quantified value of the myocardial ischemia, the cardiac function information, and the phase information.

### (Step S4-1)

The output unit 108 acquires the subject identification information and at least one of the predicted value of the result of performing the reperfusion therapy, the result of predicting the onset of the heart failure, the result of predicting the cardiac death, the result of predicting the all-cause mortality, and the result of predicting the coronary artery disease from the processing unit 106. The output unit 108 outputs the subject identification information and at least one of the predicted value of the result of performing the reperfusion therapy, the result of predicting the onset of the heart failure, the result of predicting the cardiac death, the result of predicting the all-cause mortality, and the result of predicting the coronary artery disease that have been acquired.

In the above-described embodiment, the processing unit 106 inputs the combination of the automatically quantified value of the myocardial ischemia, the cardiac function information, and the phase information of the subject and the like to the trained model 107 in which the combination of the automatically quantified value of the myocardial ischemia, the cardiac function information, and the phase information of the subject, who is a subject from whom the result of performing the reperfusion therapy, the result of the onset of the heart failure, the result of the cardiac death, the result of the all-cause mortality, and the result of the coronary artery disease have been actually acquired, and the like are stored to acquire at least one of the predicted value of the result of performing the reperfusion therapy on the subject, the result of predicting the onset of heart failure, the result of the cardiac death, the result of the all-cause mortality, and the result of the coronary artery disease. Hereinafter, the combination of the automatically quantified value of the myocardial ischemia, the cardiac function information, and the phase information related to the generation of the trained model 107 (the subject from whom the result of performing the reperfusion therapy, the result of the onset of the heart failure, the result of the cardiac death, the result of the all-cause mortality, and the result of the coronary artery disease have been actually acquired as the subject whose combination of the automatically quantified value of the myocardial ischemia, the cardiac function information, and the phase information or the like is stored) is referred to as a model target.

The creation of the trained model 107 will be described. The trained model 107 is created by a learning model creation device. That is, the learning model creation device creates the trained model 107. In addition, the diagnosis assistance device 100 may include the learning model creation device. That is, the diagnosis assistance device 100 may create the trained model 107.

FIG. 5 is a diagram showing an example of the learning model creation device according to the present embodiment. The learning model creation device 200 according to the present embodiment is implemented by a device such as a personal computer, a server, a smartphone, a tablet computer, or an industrial computer.

The learning model creation device 200 trains a learning model (a model that is the basis of the trained model 107) using a learning dataset in which the combination of the automatically quantified value of the myocardial ischemia, the cardiac function information, and the phase information of the subject of the model target or the like is used as an input sample and at least one of the result of performing the reperfusion therapy on the subject, the result of the onset of the heart failure, the result of predicting the cardiac death, the result of predicting the all-cause mortality, and the result of predicting the coronary artery disease is used as an output sample, thereby creating the trained model 107.

For example, the learning model creation device 200 uses algorithms of a convolution neural network (CNN), a recurrent neural network (RNN), a long short-term memory (LSTM), a random forest, a support vector machine (SVM), a neural network, and the like to constrict the trained model 107. The input sample is data to be input to an input layer when the learning model is trained. The output sample is data (training data) that is a correct answer to be compared with an output value output from an output, layer when the learning model is trained.

The learning model creation device 200 includes an input unit 202, a reception unit 204, a processing unit 206, an output unit 208, and a storage unit 210.

The input unit 202 inputs information. As an example, the input unit. 202 may have an operation unit such as a keyboard or a mouse. In this case, the input unit 202 inputs information according to an operation performed by a user on the operation unit. As another example, the input unit 202 may input information from an external device. The external device may be, for example, a portable storage medium. A learning dataset is input to the input unit 202.

The reception unit 204 acquires a learning dataset from the input unit 202 and receives the acquired learning dataset. The learning dataset includes an input sample and an output sample and the input sample and the output sample are paired. The learning dataset includes a plurality of pairs. The input sample may be some of the automatically quantified value of the myocardial ischemia, the cardiac function information, the phase information, and the like (sometimes referred to as a “partial combination or the like”) instead of a combination of the automatically quantified value of the myocardial ischemia, the cardiac function information, the phase information, and the like (sometimes referred to as a “total combination or the like”).

The output sample (at least one of the predicted value of the result of performing the reperfusion therapy, the result of predicting the onset of the heart failure, the result of predicting the cardiac death, the result of predicting the all-cause mortality, and the result of predicting the coronary artery disease) is acquired from the result of performing the reperfusion therapy, the result of the onset of the heart failure, the result of predicting the cardiac death, the result of predicting the all-cause mortality, and the result of predicting the coronary artery disease of the model target.

The processing unit 206 inputs an input sample to the input layer of the learning model 207 with respect to all pairs, calculates an error between the output value output from the output layer and the output sample (training data) corresponding to the input sample, changes a parameter of the learning model 207 (trains the learning model 207) so that the error is minimized, and creates the trained model 107.

The trained model 107 created as described above is received by the diagnosis assistance device 100 from the output unit 208 via a network or a medium and acquired by the processing unit 106. When the learning model creation device 200 is included in the diagnosis assistance device 100, the processing unit 106 acquires the trained model 107 from the learning model creation device 200.

All or some of the input unit 202, the reception unit 204, the processing unit 206, and the output unit 208 are functional units (hereinafter referred to as software functional units) that are implemented by a processor such as a CPU executing a program stored in the storage unit 210.

In addition, all or some of the input unit 202, the reception unit 204, the processing unit 206, and the output unit 208 may be implemented by hardware such as an LSI circuit, an ASIC, or an FPGA or may be implemented by a combination of a software function unit and hardware.

The acquisition of at least one of the predicted value of the result of performing the reperfusion therapy, the result of predicting the onset of the heart failure, the result of predicting the cardiac death, the result of predicting the all-cause mortality, and the result of predicting the coronary artery disease by the trained model 107 will be described. The processing unit 106 inputs a combination of the automatically quantified value of the myocardial ischemia, the cardiac function information, and the phase information of the subject to the trained model 107 and obtains an output value from the trained model 107. In addition, when the trained model 107 is created using a partial combination or the like as an input sample, the processing unit. 106 inputs the partial combination or the like to the trained model 107.

### (Operation of Learning Model Creation Device 200)

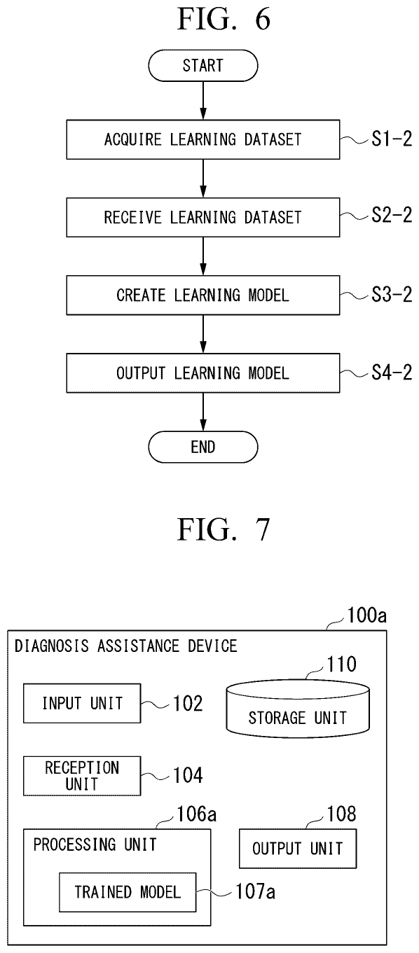

FIG. 6 is a flowchart showing an example of the operation of the learning model creation device of the present embodiment.

### (Step S1-2)

The input unit 202 acquires a learning dataset.

### (Step S2-2)

The reception unit 204 acquires the learning dataset from the input unit 202 and receives the acquired learning dataset.

### (Step S3-2)

The processing unit 206 acquires the learning dataset from the reception unit 204. The processing unit 206 inputs the input sample to the input layer of the learning model 207 with respect to all pairs of input samples and output samples included in the learning dataset, calculates an error between the output value output from the output layer and the output sample (training data) corresponding to the input sample, changes a parameter of the learning model 207 (trains the learning model 207) so that the error is minimized, and creates the trained model 107.

### (Step S4-2)

The output unit 208 acquires the learning model 207 from the processing unit 206. The output unit 208 outputs the acquired learning model 207.

A configuration in which the result of performing the reperfusion therapy is acquired by the trained model 107 may be adopted. In this case, the processing unit 106 inputs the combination of the automatically quantified value of the myocardial ischemia, the cardiac function information, and the phase information of the subject and the like to the trained model 107 in which the combination of the automatically quantified value of myocardial ischemia, the cardiac function information, and the phase information of the subject, who is a subject from whom the result of performing the reperfusion therapy has been actually acquired, and the like are stored to acquire the result of performing the reperfusion therapy on the subject. Hereinafter, the combination of the automatically quantified value of the myocardial ischemia, the cardiac function information, and the phase information related to the creation of the trained model 107 (the subject from whom the result of performing the reperfusion therapy has been actually acquired as the subject whose combination of the automatically quantified value of the myocardial ischemia, the cardiac function information, and the phase information or the like is stored) is referred to as a model target.

The creation of the trained model 107 will be described. The trained model 107 is created by the learning model creation device 200. That is, the learning model creation device 200 creates the trained model 107. In addition, the diagnosis assistance device 100 may include the learning model creation device 200. That is, the diagnosis assistance device 100 may create the trained model 107.

The learning model creation device 200 trains a learning model (a model that is the basis of the trained model 107; using a learning dataset in which the combination of the automatically quantified value of the myocardial ischemia, the cardiac function information, and the phase information of the subject of the model target or the like is used as an input sample and the result of performing the reperfusion therapy on the subject is used as an output sample, thereby creating the trained model 107. The input sample is data that is input to the input layer when the learning model is trained. The output sample is data (training data) that is the correct answer to be compared with the output value output from the output layer when the learning model is trained.

A learning dataset is input to the input unit 202. The reception unit 204 acquires the learning dataset from the input unit 202. An input sample and an output, sample are paired and the learning dataset includes a plurality of pairs. The input sample may be some of the automatically quantified value of the myocardial ischemia, the cardiac function information, the phase information, and the like (sometimes referred to as a “partial combination or the like”) instead of a combination of the automatically quantified value of the myocardial ischemia, the cardiac function information, the phase information, and the like (sometimes referred to as a “total combination or the like”).

The output sample (the result of performing the reperfusion therapy) is acquired from the result of performing the reperfusion therapy on the model target.

The processing unit 206 inputs an input sample to the input layer of the learning model 207 with respect to all pairs, calculates an error between the output value output from the output layer and the output sample (training data) corresponding to the input sample, changes a parameter of the learning model 207 (trains the learning model 207) so that the error is minimized, and creates the trained model 107.

The trained model 107 created as described above is received by the diagnosis assistance device 100 from the learning model creation device 200 via a network or a medium and acquired by the processing unit 106. When the learning model creation device 200 is included in the diagnosis assistance device 100, the processing unit 106 acquires the trained model 107 from the learning model creation device 200.

The acquisition of the result of performing the reperfusion therapy by the trained model 107 will be described. The processing unit. 106 inputs the combination of the automatically quantified value of the myocardial ischemia, the cardiac function information, and the phase information of the subject to the trained model 107 and obtains an output value from the trained model 107. In addition, when the trained model 107 is created using a partial combination or the like as an input sample, the processing unit 106 inputs the partial combination of the like to the trained model 107.

A configuration in which the result of predicting the onset of the heart failure is acquired by the trained model 107 may be adopted. In this case, the processing unit 106 inputs the combination of the automatically quantified value of the myocardial ischemia, the cardiac function information, and the phase information of the subject and the like to the trained model 107 in which the combination of the automatically quantified value of myocardial ischemia, the cardiac function information, and the phase information of the subject, who is a subject from whom the result of the onset of the heart failure has been actually acquired, and the like are stored to acquire the result of predicting the onset of the heart failure of the subject. Hereinafter, the combination of the automatically quantified value of the myocardial ischemia, the cardiac function information, and the phase information related to the creation of the trained model 107 (the subject from whom the result of the onset of the heart failure has been actually acquired as the subject whose combination of the automatically quantified value of the myocardial ischemia, the cardiac function information, and the phase information or the like is stored) is referred to as a model target.

The creation of the trained model 107 will be described. The trained model 107 is created by a learning model creation device 200. That is, the learning model creation device 200 creates the trained model 107. In addition, the diagnosis assistance device 100 may include the learning model creation device 200. That is, the diagnosis assistance device 100 may create the trained model 107.

The learning model creation device 200 trains a learning model (a model that is the basis of the trained model 107) using a learning dataset in which the combination of the automatically quantified value of the myocardial ischemia, the cardiac function information, and the phase information of the subject of the model target or the like is used as an input sample and the result of the onset of the heart failure of the subject is used as an output sample, thereby creating the trained model 107. The input sample is data that is input to the input layer when the learning model is trained. The output sample is data (training data) that is the correct answer to be compared with the output, value output from the output layer when the learning model is trained.

A learning dataset is input to the input unit 202. The reception unit 204 acquires the learning dataset from the input unit 202. An input sample and an output sample are paired and the learning dataset includes a plurality of pairs. The input sample may be some of the automatically quantified value of the myocardial ischemia, the cardiac function information, the phase information, and the like (sometimes referred to as a “partial combination or the like”) instead of a combination of the automatically quantified value of the myocardial ischemia, the cardiac function information, the phase information, and the like (sometimes referred to as a “total combination or the like”).

The output sample (the result of predicting the onset of the heart failure) is acquired from the result of the onset of the heart failure of the model target.

The processing unit 206 inputs an input sample to the input layer of the learning model 207 with respect to all pairs, calculates an error between the output value output from the output layer and the output sample (training data) corresponding to the input sample, changes a parameter of the learning model 207 (trains the learning model 207) so that the error is minimized, and creates the trained model 107.

The trained model 107 created as described above is received by the diagnosis assistance device 100 from the learning model creation device 200 via a network or a medium and acquired by the processing unit 106. When the learning model creation device 200 is included in the diagnosis assistance device 100, the processing unit 106 acquires the trained model 107 from the learning model creation device 200.

The acquisition of the result of predicting the onset of the heart failure by the trained model 107 will be described. The processing unit 106 inputs the combination of the automatically quantified value of the myocardial ischemia, the cardiac function information, and the phase information of the subject to the trained model 107 and obtains an output value from the trained model 107. In addition, when the trained model 107 is created using a partial combination of the like as an input sample, the processing unit 106 inputs the partial combination or the like to the trained model 107.

A configuration in which the result of predicting the cardiac death is acquired by the trained model 107 may be adopted. In this case, the processing unit 106 inputs the combination of the automatically quantified value of the myocardial ischemia, the cardiac function information, and the phase information of the subject and the like to the trained model 107 in which the combination of the automatically quantified value of myocardial ischemia, the cardiac function information, and the phase information of the subject, who is a subject from whom the result of the cardiac death has been actually acquired, and the like are stored to acquire the result of predicting the cardiac death of the subject. Hereinafter, the combination of the automatically quantified value of the myocardial ischemia, the cardiac function information, and the phase information related to the creation of the trained model 107 (the subject from whom the result of the cardiac death has been actually acquired as the subject whose combination of the automatically quantified value of the myocardial ischemia, the cardiac function information, and the phase information or the like is stored) is referred to as a model target.

The creation of the trained model 107 will be described. The trained model 107 is created by the learning model creation device 200. That is, the learning model creation device 200 creates the trained model 107. In addition, the diagnosis assistance device 100 may include the learning model creation device 200. That is, the diagnosis assistance device 100 may create the trained model 107.

The learning model creation device 200 trains a learning model (a model that is the basis of the trained model 107) using a learning dataset in which the combination of the automatically quantified value of the myocardial ischemia, the cardiac function information, and the phase information of the subject of the model target or the like is used as an input sample and the result of the cardiac death of the subject is used as an output sample, thereby creating the trained model 107. The input sample is data that is input to the input layer when the learning model is trained. The output sample is data (training data) that is the correct answer to be compared with the output value output from the output layer when the learning model is trained.

A learning dataset is input to the input unit 202. The reception unit 204 acquires the learning dataset from the input unit 202. An input sample and an output sample are paired and the learning dataset includes a plurality of pairs. The input sample may be some of the automatically quantified value of the myocardial ischemia, the cardiac function information, the phase information, and the like (sometimes referred so as a “partial combination or the like”) instead of a combination of the automatically quantified value of the myocardial ischemia, the cardiac function information, the phase information, and the like (sometimes referred to as a “total combination or the like”).

The output sample (the result of predicting the cardiac death) is acquired from the result of the onset of the heart failure of the model target.

The processing unit 206 inputs an input sample to the input layer of the learning model 207 with respect to all pairs, calculates an error between the output value output from the output layer and the output sample (training data) corresponding to the input sample, changes a parameter of the learning model 207 (trains the learning model 207) so that the error is minimized, and creates the trained model 107.

The trained model 107 created as described above is received by the diagnosis assistance device 100 from the learning model creation device 200 via a network or a medium and acquired by the processing unit 106. When the learning model creation device 200 is included in the diagnosis assistance device 100, the processing unit 106 acquires the trained model 107 from the learning model creation device 200.

The acquisition of the result of predicting the cardiac death by the trained model 107 will be described. The processing unit 106 inputs the combination of the automatically quantified value of the myocardial ischemia, the cardiac function information, and the phase information of the subject to the trained model 107 and obtains an output value from the trained model 107. In addition, when the trained model 107 is created using a partial combination or the like as an input sample, the processing unit 106 inputs the partial combination of the like to the trained model 107.

A configuration in which the result of predicting the all-cause mortality is acquired by the trained model 107 may be adopted. In this case, the processing unit 106 inputs the combination of the automatically quantified value of the myocardial ischemia, the cardiac function information, and the phase information of the subject and the like to the trained model 107 in which the combination of the automatically quantified value of myocardial ischemia, the cardiac function information, and the phase information of the subject, who is a subject from whom the result of the all-cause mortality has been actually acquired, and the like are stored to acquire the result of predicting the all-cause mortality of the subject. Hereinafter, the combination of the automatically quantified value of the myocardial ischemia, the cardiac function information, and the phase information related to the creation of the trained model 107 (the subject from whom the result of the all-cause mortality has been actually acquired as the subject whose combination of the automatically quantified value of the myocardial ischemia, the cardiac function information, and the phase information or the like is stored) is referred to as a model target.

The creation of the trained model 107 will be described. The trained model 107 is created by the learning model creation device 200. That is, the learning model creation device 200 creates the trained model 107. In addition, the diagnosis assistance device 100 may include the learning model creation device 200. That is, the diagnosis assistance device 100 may create the trained model 107.

The learning model creation device 200 trains a learning model (a model that is the basis of the trained model 107) using a learning dataset in which the combination of the automatically quantified value of the myocardial ischemia, the cardiac function information, and the phase information of the subject of the model target or the like is used as an input sample and the result of the all-cause mortality of the subject is used as an output sample, thereby creating the trained model 107. The input sample is data that is input to the input layer when the learning model is trained. The output sample is data (training data) that is the correct answer to be compared with the output value output from the output layer when the learning model is trained.

A learning dataset is input to the input unit 202. The reception unit 204 acquires the learning dataset from the input unit 202. An input sample and an output sample are paired and the learning dataset includes a plurality of pairs. The input sample may be some of the automatically quantified value of the myocardial ischemia, the cardiac function information, the phase information, and the like (sometimes referred to as a “partial combination or the like”) instead of a combination of the automatically quantified value of the myocardial ischemia, the cardiac function information, the phase information, and the like (sometimes referred to as a “total combination of the like”).

The output sample (the result of predicting the all-cause mortality) is acquired from the result of the onset of the heart failure of the model target.

The processing unit 206 inputs an input sample to the input layer of the learning model 207 with respect to all pairs, calculates an error between the output value output from the output layer and the output sample (training data) corresponding to the input sample, changes a parameter of the learning model 207 (trains the learning model 207) so that the error is minimized, and creates the trained model 107.

The trained model 107 created as described above is received by the diagnosis assistance device 100 from the learning model creation device 200 via a network or a medium and acquired by the processing unit 106. When the learning model creation device 200 is included in the diagnosis assistance device 100, the processing unit 106 acquires the trained model 107 from the learning model creation device 200.

The acquisition of the result of predicting the all-cause mortality by the trained model 107 will be described. The processing unit 106 inputs the combination of the automatically quantified value of the myocardial ischemia, the cardiac function information, and the phase information of the subject to the trained model 107 and obtains an output value from the trained model 107. In addition, when the trained model 107 is created using a partial combination or the like as an input sample, the processing unit 106 inputs the partial combination or the like to the trained model 107.

A configuration in which the result of predicting the coronary artery disease is acquired by the trained model 107 may be adopted. In this case, the processing unit 106 inputs the combination of the automatically quantified value of the myocardial ischemia, the cardiac function information, and the phase information of the subject and the like to the trained model 107 in which the combination of the automatically quantified value of myocardial ischemia, the cardiac function information, and the phase information of the subject, who is a subject from whom the result of the coronary artery disease has been actually acquired, and the like are stored to acquire the result of predicting the coronary artery disease of the subject. Hereinafter, the combination of the automatically quantified value of the myocardial ischemia, the cardiac function information, and the phase information related to the creation of the trained model 107 (the subject from whom the result of the coronary artery disease has been actually acquired as the subject whose combination of the automatically quantified value of the myocardial ischemia, the cardiac function information, and the phase information or the like is stored) is referred to as a model target.

The creation of the trained model 107 will be described. The trained model 107 is created by the learning model creation device 200. That is, the learning model creation device 200 creates the trained model 107. In addition, the diagnosis assistance device 100 may include the learning model creation device 200. That is, the diagnosis assistance device 100 may create the trained model 107.

The learning model creation device 200 trains a learning model (a model that is the basis of the trained model 107) using a learning dataset in which the combination of the automatically quantified value of the myocardial ischemia, the cardiac function information, and the phase information of the subject of the model target or the like is used as an input sample and the result of the coronary artery disease of the subject is used as an output sample, thereby creating the trained model 107. The input sample is data that is input to the input layer when the learning model is trained. The output sample is data (training data) that is the correct answer to be compared with the output value output from the output layer when the learning model is trained.

A learning dataset is input to the input unit 202. The reception unit 204 acquires the learning dataset from the input unit 202. An input sample and an output sample are paired and the learning dataset includes a plurality of pairs. The input sample may be some of the automatically quantified value of the myocardial ischemia, the cardiac function information, the phase information, and the like (sometimes referred to as a “partial combination or the like”) instead of a combination of the automatically quantified value of the myocardial ischemia, the cardiac function information, the phase information, and the like (sometimes referred to as a “total combination or the like”).

The output sample (the result of predicting the coronary artery disease) is acquired from the result of the onset of the heart failure of the model target.

The processing unit 206 inputs an input sample to the input layer of the learning model 207 with respect to all pairs, calculates an error between the output value output from the output layer and the output sample (training data) corresponding to the input sample, changes a parameter of the learning model 207 (trains the learning model 207) so that the error is minimized, and creates the trained model 107.

The trained model 107 created as described above is received by the diagnosis assistance device 100 from the learning model creation device 200 via a network or a medium and acquired by the processing unit 106. When the learning model creation device 200 is included in the diagnosis assistance device 100, the processing unit 106 acquires the trained model 107 from the learning model creation device 200.

The acquisition of the result of predicting the coronary artery disease by the trained model 107 will be described. The processing unit 106 inputs the combination of the automatically quantified value of the myocardial ischemia, the cardiac function information, and the phase information of the subject to the trained model 107 and obtains an output value from the trained model 107. In addition, when the trained model 107 is created using a partial combination or the like as an input sample, the processing unit 106 inputs the partial combination or the like to the trained model 107.

Although the diagnosis assistance device 100 receives subject-related information and acquires at least one of the predicted value of the result of performing the reperfusion therapy, the result of predicting the onset of the heart failure, the result of predicting the cardiac death, the result of predicting the all-cause mortality, and the result of predicting the coronary artery disease on the basis of the automatically quantified value of the myocardial ischemia, the cardiac function information, and the phase information included in the received subject-related information and the trained model 107 in the above-described embodiment, the present invention is not limited to the above example. For example, the diagnosis assistance device 100 may be configured to receive the subject-related information and acquire a result of predicting the implementation of the reperfusion therapy in addition to at least one of the predicted value of the result of performing the reperfusion therapy, the result of predicting the onset of the heart failure, the result of predicting the cardiac death, the result of predicting the all-cause mortality, and the result of predicting the coronary artery disease on the basis of the automatically quantified value of the myocardial ischemia, the cardiac function information, and the phase information included in the received subject-related information and the trained model 107.

Predicting the implementation of the reperfusion therapy is predicting the need for emergency coronary artery treatment for acute near-myocardial infarction or the need for elective PCI or bypass surgery based on the presence of a non-urgent obvious coronary artery lesion.

According to the diagnosis assistance device of the present embodiment, because the diagnosis assistance device 100 can receive the automatically quantified value of the myocardial ischemia of the subject and the cardiac function information and the phase information acquired when the stress myocardial scintigraphy is performed on the subject and can acquire at least one of the predicted value of the result of performing the reperfusion therapy, the result of predicting the onset of the heart failure, the result of predicting the cardiac death, the result of predicting the all-cause mortality, and the result of predicting the coronary artery disease on the basis of the automatically quantified value of the myocardial ischemia, the cardiac function information, and the phase information that have been received, and a trained model, it is possible to acquire at least one of the predicted value of the result of performing the reperfusion therapy, the result of predicting the onset of the heart failure, the result of predicting the cardiac death, the result of predicting the all-cause mortality, and the result of predicting the coronary artery disease for the subject, without relying on human visual inspection.

The diagnosis assistance device 100 requires less computational cost than machine learning for processing images. The method is a method of making the determination according to a machine learning technique using numerical information routinely obtained at facilities where myocardial scintigraphy is performed without using an algorithm for processing clinical images, and can be implemented in a state of separation from an image reading system or a reporting system.

The phase information is obtained by capturing an increase or a decrease in a gamma ray count in a region of interest on the image associated with the contraction and expansion of a heart as phase information using video information from an electrocardiogram-gated imaging method such as electrocardiogram-gated myocardial scintigraphy. The phase information detects myocardial contraction phase information including a position (coordinates) of the myocardium corresponding to a site of each segment of the myocardium (an anterior wall, an inferior wall, a side wall, or a septum). The phase information is created as secondary information in the form of a histogram in which an x-axis is a time axis of a cardiac cycle (from the R wave to the next R wave on the electrocardiogram) and a vertical axis represents the number of myocardial segments that have reached the end of the contraction in the phase on the x-axis. Because the phase information detects changes in the wall thickness of the left ventricular myocardium, elements, which cannot be detected by the electrocardiogram, such as wall motion abnormalities during myocardial ischemia are included therein.

According to the learning model creation device of the present embodiment, the learning model creation device 200 can create a learning model by performing machine learning on a relationship of the automatically quantified value of the myocardial ischemia, the cardiac function information, the phase information, and at least one of the result of performing the reperfusion therapy, the result of the onset of the heart failure, the result of the cardiac death, the result of the all-cause mortality, and the result of the coronary artery disease using the automatically quantified value of the myocardial ischemia, the cardiac function information, and the phase information as explanatory variables and using at least one of the result of performing the reperfusion therapy, the result of the onset of the heart failure, the result of the cardiac death, the result of the all-cause mortality, and the result of the coronary artery disease as an objective variable on the basis of the learning dataset.

### Modified Example 1 of Embodiment

A diagnosis assistance device 100a according to Modified Example 1 of the embodiment will be described.

FIG. 7 is a diagram showing an example of a diagnosis assistance device according to Modified Example 1 of the embodiment.

Unlike the diagnosis assistance device 100 of the embodiment, the diagnosis assistance device 100a of Modified Example 1 of the embodiment receives subject-related information further including at least one of an index for predicting the onset of coronary artery disease acquired before stress myocardial scintigraphy is performed on a subject, a coronary artery calcium score of the subject acquired when the stress myocardial scintigraphy is performed, a body mass index of the subject, and a left ventricular volume ratio of the subject during stress and rest.

The diagnosis assistance device 100a obtains at least one of a predicted value of a result of performing reperfusion therapy, a result of predicting the onset of heart failure, a result of predicting cardiac death, a result of predicting all-cause mortality, and a result of predicting coronary artery disease on the basis of at least one of the index for predicting the onset of coronary artery disease, the coronary artery calcium score, the body mass index, and the left ventricular volume ratio, an automatically quantified value of myocardial ischemia, cardiac function information, and phase information included in the received subject-related information and a trained model.

Here, the trained model is obtained by performing machine learning on a relationship between a combination of at least one of the index for predicting the onset of the coronary artery disease, the coronary artery calcium score, the body mass index, and the left ventricular volume ratio, the automatically quantified value of the myocardial ischemia, the cardiac function information, and the phase information and at least one of the predicted value of the result of performing the reperfusion therapy, the result of predicting the onset of the heart failure, the result of predicting the cardiac death, the result of predicting the all-cause mortality, and the result of predicting the coronary artery disease.

The diagnosis assistance device 100a outputs subject identification information and at least one of the predicted value of the result of performing the reperfusion therapy, the result of predicting the onset of the heart failure, the result of predicting the cardiac death, the result of predicting the all-cause mortality, and the result of predicting the coronary artery disease that have been acquired.

The diagnosis assistance device 100a is implemented by a device such as a personal computer, a server, a smartphone, a tablet computer, or an industrial computer. The diagnosis assistance device 100a includes an input unit 102, a reception unit 104, a processing unit 106a, an output unit 108, and a storage unit 110.

The reception unit 104 acquires the subject-related information from the input unit 102. The reception unit. 104 acquires the subject identification information included in the acquired subject-related information, at least one of the index for predicting the onset of the coronary artery disease, the coronary artery calcium score, the body mass index, and the left ventricular volume ratio, the automatically quantified value of the myocardial ischemia, the cardiac function information, and the phase information and receives the subject identification information, at least one of the index for predicting the onset of the coronary artery disease, the coronary artery calcium score, the body mass index, and the left ventricular volume ratio, the automatically quantified value of the myocardial ischemia, the cardiac function information, and the phase information that have been acquired.

An example of the index for predicting the onset of the coronary artery disease is a Suita score. The Suita score is a score table that shows a likelihood of experiencing a myocardial infarction. The Suita score is referred to as a coronary artery disease onset prediction model and it is possible to determine whether or not there are lipid abnormalities having a strong influence on coronary artery disease such as angina pectoris or myocardial infarction and investigate the extent to which coronary artery disease occurs on the basis of data on Japanese people.

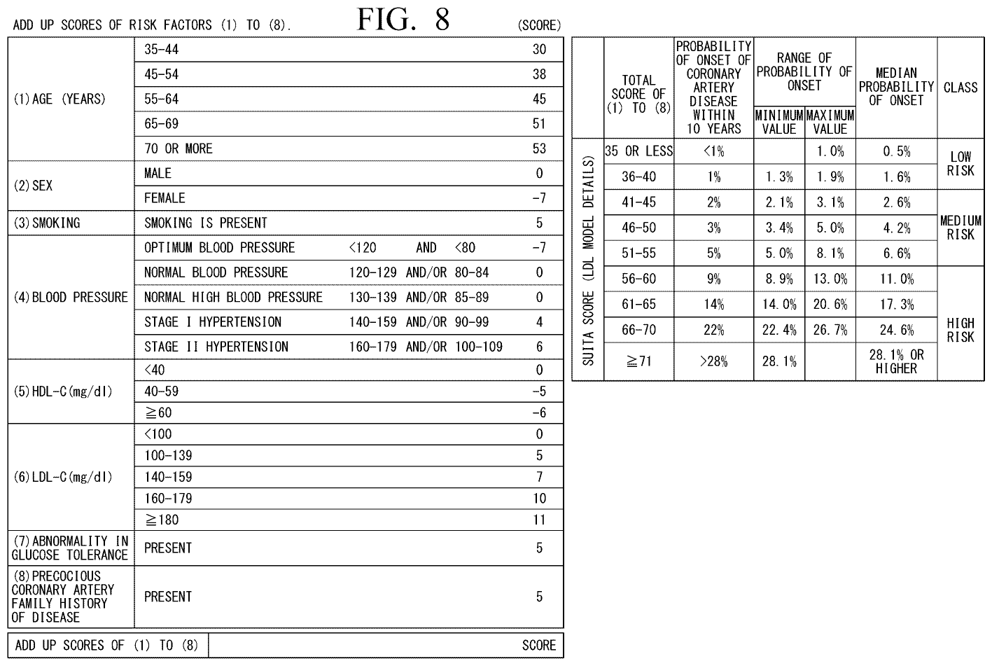

FIG. 8 is an explanatory diagram of an example of the Suita score. The Suita score is calculated on the basis of risk factors. Examples of risk factors include (1) age, (2) sex, (3) presence or absence of smoking. (4) blood pressure, (5) HDL cholesterol value, (6) LDL cholesterol value, (7) presence or absence of abnormality in glucose tolerance, (8) presence or absence of family history of premature coronary artery disease, and the like. For each of a plurality of risk factors, a score is associated with the answer to the risk factor. The Suita score is derived by adding ap the scores corresponding to the subject's answers for each of the multiple risk factors. The Suita score is used to derive a probability of coronary artery disease occurring within 10 years, a range of the onset probability, and a median of the onset probability, and the risk of the subject is classified. Hereinafter, the description of a case where the Suita score is applied as an example of the index for predicting the onset of the coronary artery disease will be continued.

The coronary artery calcium score is calculated from a coronary artery calcium scan. The coronary artery calcium scan is a test in which calcification of the coronary artery from a computed tomography (CT) image captured according to non-contrast electrocardiogram-gated imaging is quantitatively assessed. The coronary artery calcium score is useful for predicting the risk of cardiac events and the risk of developing ischemic heart disease. The description will continue with reference back to FIG. 7.

A body mass index (BMI) is a body mass index that indicates a degree of obesity in humans and is used as an index for determining obesity. The BMI is calculated by weight (kg)÷height (m)÷height (m), and the Japan Society for the Study of Obesity defines a BMI of 25 or more as obesity. The higher the BMI, the higher the risk of developing high blood pressure, diabetes, and dyslipidemia. However, it is known that a low BMI can also cause health problems, and it is said that the healthiest is around the standard weight of BMI 22. In cases of a high BMI value, gamma rays emitted from the myocardium to the outside during stress myocardial scintigraphy may be absorbed and attenuated by fat tissue and the like, which may lead to a decrease in image quality and a decrease in diagnostic accuracy.

The left ventricular volume ratio is calculated from a ratio of left ventricular volumes between stress and rest based on images during stress and rest captured in stress myocardial scintigraphy. It has been reported that the TID ratio is high in severe coronary artery disease, such as multivessel disease or main trunk disease, and it is known as a diagnostic auxiliary index that complements the weaknesses of myocardial scintigraphy.

The processing unit 106a acquires the subject identification information, at least one of the index for predicting the onset of the coronary artery disease, the coronary artery calcium score, the body mass index, and the left ventricular volume ratio, the automatically quantified value of the myocardial ischemia, the cardiac function information, and the phase information from the reception unit 104. The processing unit 106a includes a trained model 107a.

The trained model 107a is obtained by performing machine learning on a relationship between a combination of at least one of the index for predicting the onset of the coronary artery disease, the coronary artery calcium score, the body mass index, and the left ventricular volume ratio, the automatically quantified value of the myocardial ischemia, the cardiac function information, and the phase information and at least one of the predicted value of the result of performing the reperfusion therapy, the result of predicting the onset of the heart failure, the result of predicting the cardiac death, the result of predicting the all-cause mortality, and the result of predicting the coronary artery disease.

The processing unit 106a inputs a combination of at least one of the index for predicting the onset of the coronary artery disease, the coronary artery calcium score, the body mass index, and the left ventricular volume ratio, the automatically quantified value of the myocardial ischemia, the cardiac function information, and the phase information that have been acquired to the trained model 107a and acquires at least one of the predicted value of the result of performing the reperfusion therapy, the result of predicting the onset of the heart failure, the result of predicting the cardiac death, the result of predicting the all-cause mortality, and the result of predicting the coronary artery disease output by the trained model 107a with respect to the combination of at least one of the index for predicting the onset of the coronary artery disease, the coronary artery calcium score, the body mass index, and the left ventricular volume ratio, the automatically quantified value of the myocardial ischemia, the cardiac function information, and the phase information.

All or a part of the processing unit 106a is a functional unit (hereinafter referred to as a software functional unit) that is implemented by a processor such as a CPU executing a program stored in the storage unit 110. In addition, all or a part of the processing unit 106a may be implemented by hardware such as an LSI circuit, an ASIC, or an FPGA or may be implemented by a combination of a software functional unit and hardware.

### (Operation of Diagnosis Assistance Device 100a)

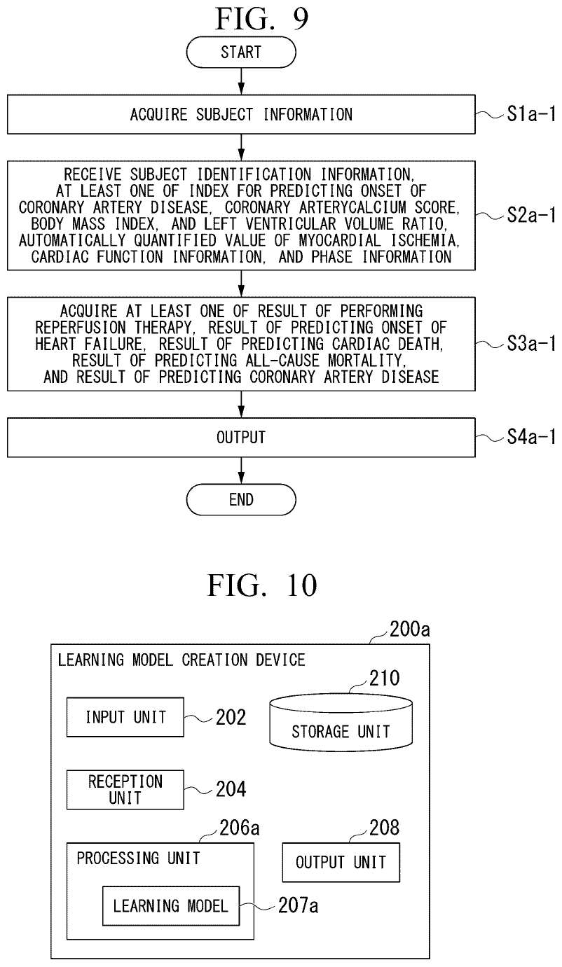

FIG. 9 is a flowchart showing an example of an operation of a diagnosis assistance device according to Modified Example 1 of the embodiment.

### (Step S1a-1)

The input unit 102 acquires subject-related information.

### (Step S2a-1)

The reception unit 104 acquires the subject-related information from the input unit 102. The reception unit 104 acquires subject identification information included in the acquired subject-related information, at least one of an index for predicting the onset of coronary artery disease, a coronary artery calcium score, a body mass index, and a left ventricular volume ratio, an automatically quantified value of myocardial ischemia, cardiac function information, and phase information and receives the subject identification information, at least one of the index for predicting the onset of the coronary artery disease, the coronary artery calcium score, the body mass index, and the left ventricular volume ratio, the automatically quantified value of the myocardial ischemia, the cardiac function information, and the phase information that have been acquired.

### (Step S3a-1)

The processing unit 106a acquires the subject identification information, at least one of the index for predicting the onset of the coronary artery disease, the coronary artery calcium score, the body mass index, and the left ventricular volume ratio, the automatically quantified value of the myocardial ischemia, the cardiac function information, and the phase information from the reception unit 104. The processing unit 106a inputs a combination of at least one of the index for predicting the onset of the coronary artery disease, the coronary artery calcium score, the body mass index, and the left ventricular volume ratio, the automatically quantified value of the myocardial ischemia, the cardiac function information, and the phase information that have been acquired to the trained model 107a and acquires at least one of the predicted value of the result of performing the reperfusion therapy, the result of predicting the onset of the heart failure, the result of predicting the cardiac death, the result of predicting the all-cause mortality, and the result of predicting the coronary artery disease output by the trained model 107a with respect to at least one of the index for predicting the onset of the coronary artery disease, the coronary artery calcium score, the body mass index, and the left ventricular volume ratio, the automatically quantified value of the myocardial ischemia, the cardiac function information, and the phase information that have been input.

### (Step S4a-1)

The output unit 108 acquires the subject identification information and at least one of the predicted value of the result of performing the reperfusion therapy, the result of predicting the onset of the heart failure, the result of predicting the cardiac death, the result of predicting the all-cause mortality, and the result of predicting the coronary artery disease from the processing unit 106a. The output unit 108 outputs the subject identification information and at least one of the predicted value of the result of performing the reperfusion therapy, the result of predicting the onset of the heart failure, the result of predicting the cardiac death, the result of predicting the all-cause mortality, and the result of predicting the coronary artery disease that have been acquired.

In Modified Example 1 of the embodiment described above, the processing unit 106a inputs the combination of at least one of the index for predicting the onset of the coronary artery disease, the coronary artery calcium score, the body mass index, and the left ventricular volume ratio, the automatically quantified value of the myocardial ischemia, the cardiac function information, and the phase information of the subject to the trained model 107a in which the combination of at least one of the index for predicting the onset of the coronary artery disease, the coronary artery calcium score, the body mass index, and the left ventricular volume ratio, the automatically quantified value of the myocardial ischemia, the cardiac function information, and the phase information of the subject, who is a subject from whom at least one of the result of performing the reperfusion therapy, the result of the onset of the heart failure, the result of predicting the cardiac death, the result of predicting the all-cause mortality, and the result of predicting the coronary artery disease has been actually acquired, and the like are stored to acquire at least one of the predicted value of the result of performing the reperfusion therapy on the subject, the result of predicting the onset of the heart failure, the result of predicting the cardiac death, the result of predicting the all-cause mortality, and the result of predicting the coronary artery disease.

Hereinafter, the combination of at least one of the index for predicting the onset of the coronary artery disease, the coronary artery calcium score, the body mass index, and the left ventricular volume ratio, the automatically quantified value of the myocardial ischemia, the cardiac function information, and the phase information related to the generation of the trained model 107a (the subject from whom at least one of the result of performing the reperfusion therapy, the result of the onset of the heart failure, the result of predicting the cardiac death, the result of predicting the all-cause mortality, and the result of predicting the coronary artery disease has been actually acquired as the subject whose combination of at least one of the index for predicting the onset of the coronary artery disease, the coronary artery calcium score, the body mass index, and the left ventricular volume ratio, the automatically quantified value of the myocardial ischemia, the cardiac function information, and the phase information or the like is stored) is referred to as a model target.

The generation of the trained model 107a will be described. The trained model 107a is created by a learning model creation device. That is, the model creation device creates the trained model 107a. In addition, the diagnosis assistance device 100a may include the model creation device. That is, the diagnosis assistance device 100a may create the trained model 107a.

FIG. 10 is a diagram showing an example of a learning model creation device according to Modified Example 1 of the embodiment. The learning model creation device 200a according to Modified Example 1 of the embodiment is implemented by a device such as a personal computer, a server, a smartphone, a tablet computer, or an industrial computer.

The learning model creation device 200a trains a learning model (a model that is the basis of the trained model 107a) using a learning dataset in which the combination of at least one of the index for predicting the onset of the coronary artery disease, the coronary artery calcium score, the body mass index, and the left ventricular volume ratio, the automatically quantified value of the myocardial ischemia, the cardiac function information, and the phase information of the subject of the model target or the like is used as an input sample and at least one of the result of performing the reperfusion therapy on the subject, the result of the onset of the heart failure, the result of predicting the cardiac death, the result of predicting the all-cause mortality, and the result of predicting the coronary artery disease is used as an output sample, thereby creating the trained model 107a.

For example, the learning model creation device 200a uses algorithms of a CNN, an RNN, an LSTM, a random forest, an SVM, a neural network, and the like to construct the trained model 107a. The input sample is data to be input to an input layer when the learning model is trained. The output sample is data (training data) that is a correct answer to be compared with an output value output from an output layer when the learning model is trained.

The learning model creation device 200a includes an input unit 202, a reception unit 204, a processing unit 206a, an output unit 208, and a storage unit 210.

A learning dataset is input to the input unit 202. The reception unit 204 acquires the learning dataset from the input unit 202. An input sample and an output sample are paired, and the learning dataset includes a plurality of pairs. The input sample may be some of at least one of the index for predicting the onset of the coronary artery disease, the coronary artery calcium score, the body mass index, and the left ventricular volume ratio, the automatically quantified value of the myocardial ischemia, the cardiac function information, the phase information, and the like (sometimes referred to as a “partial combination or the like”) instead of a combination of at least one of the index for predicting the onset of the coronary artery disease, the coronary artery calcium score, the body mass index, and the left ventricular volume ratio, the automatically quantified value of the myocardial ischemia, the cardiac function information, the phase information, and the like (sometimes referred to as a “total combination or the like”).

The output sample (at least one of the predicted value of the result of performing the reperfusion therapy, the result of predicting the onset of the heart failure, the result of predicting the cardiac death, the result of predicting the all-cause mortality, and the result of predicting the coronary artery disease is acquired from at least one of the result of performing the reperfusion therapy, the result of the onset of the heart failure, the result of predicting the cardiac death, the result of predicting the all-cause mortality, and the result of predicting the coronary artery disease of the model target.

The learning model creation device 200a inputs an input sample to the input layer of the learning model 207a with respect to all pairs, calculates an error between the output value output from the output layer and the output sample (training data) corresponding to the input sample, and changes a parameter of the learning model 207a (trains the learning model 207a) so that the error is minimized, and creates a trained model 107a.

The trained model 107a created as described above is received by the diagnosis assistance device 100a from the output unit 208 via a network or a medium and acquired by the processing unit. 106a. When the learning model creation device 200a is included in the diagnosis assistance device 100a, the processing unit 106a acquires the trained model 107a from the learning model creation device 200a.

All or a part of the processing unit 206a is a functional unit (hereinafter referred to as a software functional unit) that is implemented by a processor such as a CPU executing a program stored in the storage unit 210. In addition, all or a part of the processing unit 206a may be implemented by hardware such as an LSI circuit, an ASIC, or an FPGA or may be implemented by a combination of a software functional unit and hardware.

The acquisition of at least one of the predicted value of the result of performing the reperfusion therapy, the result of predicting the onset of the heart failure, the result of predicting the cardiac death, the result of predicting the all-cause mortality, and the result of predicting the coronary artery disease by the trained model 107a will be described. The processing unit 106a inputs a combination of at least one of the index for predicting the onset of the coronary artery disease, the coronary artery calcium score, the body mass index, and the left ventricular volume ratio, the automatically quantified value of the myocardial ischemia, the cardiac function information, and the phase information of the subject to the trained model 107a and obtains an output value from the trained model 107a.

In addition, when the trained model 107a is created using a partial combination or the like as an input sample, the processing unit 106a inputs the partial combination or the like to the trained model 107a.

### (Operation of Learning Model Creation Device 200a)

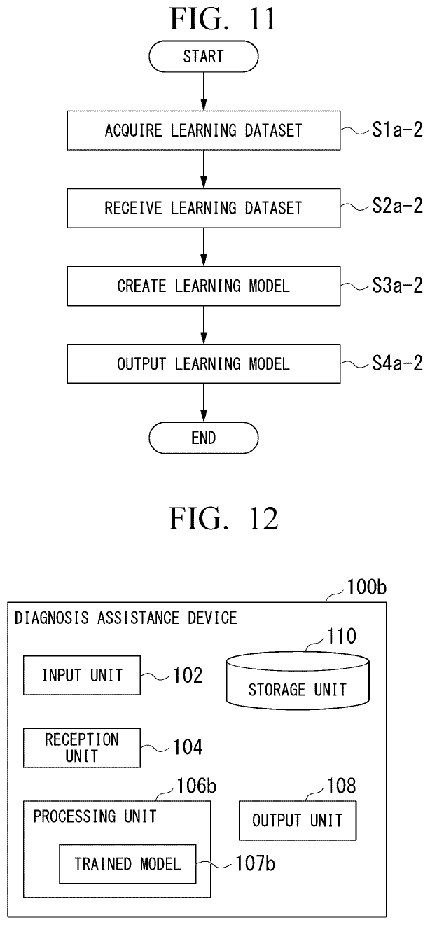

FIG. 11 is a flowchart showing an example of an operation of the learning model creation device according to Modified Example 1 of the embodiment.

### (Step S1a-2)

The input unit 202 acquires a learning dataset.

### (Step S2a-2)

The reception unit 204 acquires the learning dataset from the input unit 202. The reception unit 204 receives the acquired learning dataset.

### (Step S3a-2)

The processing unit 206a acquires the learning dataset from the reception unit 204. The processing unit 206a inputs the input sample to the input layer of the learning model 207a with respect to all pairs of input samples and output samples included in the learning dataset, calculates an error between the output value output from the output layer and the output sample (training data) corresponding to the input sample, changes a parameter of the learning model 207a (trains the learning model 207a) so that the error is minimized, and creates the trained model 107a.

### (Step S4a-2)

The output unit 208 acquires the learning model 207a from the processing unit 206a and outputs the acquired learning model 207a.

A configuration in which the result of performing the reperfusion therapy is acquired by the trained model 107a may be adopted. In this case, the processing unit 106a inputs the combination of at least one of the index for predicting the onset of the coronary artery disease, the coronary artery calcium score, the body mass index, and the left ventricular volume ratio, the automatically quantified value of the myocardial ischemia, the cardiac function information, and the phase information of the subject and the like to the trained model 107a in which the combination of at least one of the index for predicting the onset of the coronary artery disease, the coronary artery calcium score, the body mass index, and the left ventricular volume ratio, the automatically quantified value of the myocardial ischemia, the cardiac function information, and the phase information of the subject, who is a subject from which the result of performing the reperfusion therapy has been actually acquired, and the like are stored to acquire the result of performing the reperfusion therapy on the subject.

Hereinafter, the combination of at least one of the index for predicting the onset of the coronary artery disease, the coronary artery calcium score, the body mass index, and the left ventricular volume ratio, the automatically quantified value of the myocardial ischemia, the cardiac function information, and the phase information related to the creation of the trained model 107a (a subject from whom the result of performing the reperfusion therapy has been actually acquired as a subject whose combination of at least one of the index for predicting the onset of the coronary artery disease, the coronary artery calcium score, the body mass index, and the left ventricular volume ratio, the automatically quantified value of the myocardial ischemia, the cardiac function information, and the phase information of the subject or the like is stored) is referred to as a model target.

The creation of the trained model 107a will be described. The trained model 107a is created by the learning model creation device 200a. That is, the learning model creation device 200a creates the trained model 107a. In addition, the diagnosis assistance device 100a may include the learning model creation device 200a. That is, the diagnosis assistance device 100a may create the trained model 107a.

The learning model creation device 200a trains a learning model (a model that is the basis of the trained model 107a) using a learning dataset in which the combination of at least one of the index for predicting the onset of the coronary artery disease, the coronary artery calcium score, the body mass index, and the left ventricular volume ratio, the automatically quantified value of the myocardial ischemia, the cardiac function information, and the phase information of the subject of the model target or the like is used as an input sample and the result of performing the reperfusion therapy on the subject is used as an output sample, thereby creating the trained model 107a. The input sample is data that is input to the input layer when the learning model is trained. The output sample is data (training data) that is the correct answer to be compared with the output value output from the output layer when the learning model is trained.

A learning dataset is input to the input unit 202. The reception unit 204 acquires the learning dataset from the input unit 202. The input sample and the output sample are paired and the learning dataset includes a plurality of pairs. The input sample may be some of at least one of the index for predicting the onset of the coronary artery disease, the coronary artery calcium score, the body mass index, and the left ventricular volume ratio, the automatically quantified value of the myocardial ischemia, the cardiac function information, the phase information, and the like (sometimes referred to as a “partial combination or the like”) instead of a combination of at least one of the index for predicting the onset of the coronary artery disease, the coronary artery calcium score, the body mass index, and the left ventricular volume ratio, the automatically quantified value of the myocardial ischemia, the cardiac function information, the phase information, and the like (sometimes referred to as a “total combination or the like”). The output sample (the result of performing the reperfusion therapy) is acquired from the result of performing the reperfusion therapy of the model target.

The processing unit 206a inputs an input sample to the input layer of the learning model 207a with respect to all pairs, calculates an error between the output value output from the output layer and the output sample (training data) corresponding to the input sample, and changes a parameter of the learning model 207a (trains the learning model 207a) so that the error is minimized, and creates a trained model 107a.

The trained model 107a created as described above is received by the diagnosis assistance device 100a from the learning model creation device 200a via a network or a medium and acquired by the processing unit 106a. When the learning model creation device 200a is included in the diagnosis assistance device 100a, the processing unit 106a acquires the trained model 107a from the learning model creation device 200a.

The acquisition of the result of performing the reperfusion therapy by the trained model 107a will be described. The processing unit 106a inputs a combination of at least one of the index for predicting the onset of the coronary artery disease, the coronary artery calcium score, the body mass index, and the left ventricular volume ratio, the automatically quantified value of the myocardial ischemia, the cardiac function information, and the phase information of the subject to the trained model 107a and obtains an output value from the trained model 107a. In addition, when the trained model 107a is created using a partial combination or the like as an input sample, the processing unit 106a inputs the partial combination or the like to the trained model 107a.

A configuration in which the result of predicting the onset of the heart failure is acquired by the trained model 107a may be adopted. In this case, the processing unit. 106a inputs the combination of at least one of the index for predicting the onset of the coronary artery disease, the coronary artery calcium score, the body mass index, and the left ventricular volume ration, the automatically quantified value of the mycoardial ischemia, the cardiac function information, and the phase information of the subject and the like to the trained model 107a in which the combination of at least one of the index for predicting the onset of the coronary artery disease, the coronary artery calcium score, the body mass index, and the left ventricular volume ratio, the automatically quantified value of the myocardial ischemia, the cardiac function information, and the phase information of the subject, who is a subject from which the result of the onset of the heart failure has been actually acquired, and the like are stored to acquire the result of predicting the onset of the heart failure of the subject.

Hereinafter, the combination of at least one of the index for predicting the onset of the coronary artery disease, the coronary artery calcium score, the body mass index, and the left ventricular volume ratio, the automatically quantified value of the myocardial ischemia, the cardiac function information, and the phase information related to the creation of the trained model 107a (a subject from whom the result of the onset of the heart failure has been actually acquired as a subject whose combination of at least one of the index for predicting the onset of the coronary artery disease, the coronary artery calcium score, the body mass index, and the left ventricular volume ratio, the automatically quantified value of the myocardial ischemia, the cardiac function information, and the phase information or the like is stored) is referred to as a model target.

The creation of the trained model 107a will be described. The trained model 107a is created by a learning model creation device. That is, the learning model creation device creates the trained model 107a. In addition, the diagnosis assistance device 100a may include the learning model creation device. That is, the diagnosis assistance device 100a may create the trained model 107a.

The learning model creation device 200a trains a learning model (a model that is the basis of the trained model 107a) using a learning dataset in which the combination of at least one of the index for predicting the onset of the coronary artery disease, the coronary artery calcium score, the body mass index, and the left ventricular volume ratio, the automatically quantified value of the myocardial ischemia, the cardiac function information, and the phase information of the subject of the model target or the like is used as an input sample and the result of the onset of the heart failure of the subject is used as an output sample, thereby creating the trained model 107a. The input sample is data that is input to the input layer when the learning model is trained. The output sample is data (training data) that is the correct answer to be compared with the output value output from the output layer when the learning model is trained.

A learning dataset is input to the input unit 202. The reception unit 204 acquires the learning dataset from the input unit 202. The input sample and the output sample are paired and the learning dataset includes a plurality of pairs. The input sample may be some of at least one of the index for predicting the onset of the coronary artery disease, the coronary artery calcium score, the body mass index, and the left ventricular volume ratio, the automatically quantified value of the myocardial ischemia, the cardiac function information, the phase information, and the like (sometimes referred to as a “partial combination or the like”) instead of a combination of at least one of the index for predicting the onset of the coronary artery disease, the coronary artery calcium score, the body mass index, and the left ventricular volume ratio, the automatically quantified value of the myocardial ischemia, the cardiac function information, the phase information, and the like (sometimes referred to as a “total combination or the like”).

The output sample (the result of predicting the onset of the heart failure) is acquired from the result of the onset of the heart failure of the model target.

The processing unit 206a inputs an input sample to the input layer of the learning model 207a with respect to all pairs, calculates an error between the output value output from the output layer and the output sample (training data) corresponding to the input sample, and changes a parameter of the learning model 207a (trains the learning model 207a) so that the error is minimized, and creates a trained model 107a.

The trained model 107a created as described above is received by the diagnosis assistance device 100a from the learning model creation device 200a via a network or a medium and acquired by the processing unit 106a. When the learning model creation device 200a is included in the diagnosis assistance device 100a, the processing unit 106a acquires the trained model 107a from the learning model creation device 200a.

The acquisition of the result of predicting the onset of the heart failure by the trained model 107a will be described. The processing unit 106a inputs a combination of at least one of the index for predicting the onset of the coronary artery disease, the coronary artery calcium score, the body mass index, and the left ventricular volume ratio, the automatically quantified value of the myocardial ischemia, the cardiac function information, and the phase information of the subject to the trained model 107a and obtains an output value from the trained model 107a. In addition, when the trained model 107a is created using a partial combination or the like as an input sample, the processing unit 106a inputs the partial combination or the like to the trained model 107a.

A configuration in which the result of predicting the cardiac death is acquired by the trained model 107a may be adopted. In this case, the processing unit 106a inputs the combination of at least one of the index for predicting the onset of the coronary artery disease, the coronary artery calcium score, the body mass index, and the left ventricular volume ratio, the automatically quantified value of the myocardial ischemia, the cardiac function information, and the phase information of the subject and the like to the trained model 107a in which the combination of at least one of the index for predicting the onset of the coronary artery disease, the coronary artery calcium score, the body mass index, and the left ventricular volume ratio, the automatically quantified value of the myocardial ischemia, the cardiac function information, and the phase information of the subject, who is a subject from which the result of the cardiac death has been actually acquired, and the like are stored to acquire the result of predicting the cardiac death of the subject.

Hereinafter, the combination of at least one of the index for predicting the onset of the coronary artery disease, the coronary artery calcium score, the body mass index, and the left ventricular volume ratio, the automatically quantified value of the myocardial ischemia, the cardiac function information, and the phase information related to the creation of the trained model 107a (a subject from whom the result of the cardiac death has been actually acquired as a subject whose combination of at least one of the index for predicting the onset of the coronary artery disease, the coronary artery calcium score, the body mass index, and the left ventricular volume ratio, the automatically quantified value of the myocardial ischemia, the cardiac function information, and the phase information of the subject or the like is stored) is referred to as a model target.

The creation of the trained model 107a will be described. The trained model 107a is created by a learning model creation device. That is, the learning model creation device creates the trained model 107a. In addition, the diagnosis assistance device 100a may include the learning model creation device. That is, the diagnosis assistance device 100a may create the trained model 107a.

The learning model creation device 200a trains a learning model (a model that is the basis of the trained model 107a) using a learning dataset in which the combination of at least one of the index for predicting the onset of the coronary artery disease, the coronary artery calcium score, the body mass index, and the left ventricular volume ratio, the automatically quantified value of the myocardial ischemia, the cardiac function information, and the phase information of the subject of the model target or the like is used as an input sample and the result of the cardiac death of the subject is used as an output sample, thereby creating the trained model 107a. The input sample is data that is input to the input layer when the learning model is trained. The output sample is data (training data) that is the correct answer to be compared with the output value output from the output layer when the learning model is trained.

A learning dataset is input to the input unit 202. The reception unit 204 acquires the learning dataset from the input unit 202. The input sample and the output sample are paired and the learning dataset includes a plurality of pairs. The input sample may be some of at least one of the index for predicting the onset of the coronary artery disease, the coronary artery calcium score, the body mass index, and the left ventricular volume ratio, the automatically quantified value of the myocardial ischemia, the cardiac function information, the phase information, and the like (sometimes referred to as a “partial combination or the like”) instead of a combination of at least one of the index for predicting the onset of the coronary artery disease, the coronary artery calcium score, the body mass index, and the left ventricular volume ratio, the automatically quantified value of the myocardial ischemia, the cardiac function information, the phase information, and the like (sometimes referred to as a “total combination or the like”).

The output sample (the result of predicting the cardiac death) is acquired from the result of the cardiac death of the model target.

The processing unit 206a inputs an input sample to the input layer of the learning model 207a with respect to all pairs, calculates an error between the output value output from the output layer and the output sample (training data) corresponding to the input sample, and changes a parameter of the learning model 207a (trains the learning model 207a) so that the error is minimized, and creates a trained model 107a.

The trained model 107a created as described above is received by the diagnosis assistance device 100a from the learning model creation device 200a via a network or a medium and acquired by the processing unit 106a. When the learning model creation device 200a is included in the diagnosis assistance device 100a, the processing unit 106a acquires the trained model 107a from the learning model creation device 200a.

The acquisition of the result of predicting the cardiac death by the trained model 107a will be described. The processing unit 106a inputs a combination of at least one of the index for predicting the onset of the coronary artery disease, the coronary artery calcium score, the body mass index, and the left ventricular volume ratio, the automatically quantified value of the myocardial ischemia, the cardiac function information, and the phase information of the subject to the trained model 107a and obtains an output value from the trained model 107a. In addition, when the trained model 107a is created using a partial combination or the like as an input sample, the processing unit 106a inputs the partial combination or the like to the trained model 107a.

A configuration in which the result of predicting the all-cause mortality is acquired by the trained model 107a may be adopted. In this case, the processing unit. 106a inputs the combination of at least one of the index for predicting the onset of the coronary artery disease, the coronary artery calcium score, the body mass index, and the left ventricular volume ratio, the automatically quantified value of the myocardial ischemia, the cardiac function information, and the phase information of the subject and the like to the trained model 107a in which the combination of at least one of the index for predicting the onset of the coronary artery disease, the coronary artery calcium score, the body mass index, and the left ventricular volume ratio, the automatically quantified value of the myocardial ischemia, the cardiac function information, and the phase information of the subject, who is a subject from which the result of the all-cause mortality has been actually acquired, and the like are stored to acquire the result of predicting the all-cause mortality of the subject.

Hereinafter, the combination of at least one of the index for predicting the onset of the coronary artery disease, the coronary artery calcium score, the body mass index, and the left ventricular volume ratio, the automatically quantified value of the myocardial ischemia, the cardiac function information, and the phase information related to the creation of the trained model 107a (a subject from whom the result of the all-cause mortality has been actually acquired as a subject whose combination of at least one of the index for predicting the onset of the coronary artery disease, the coronary artery calcium score, the body mass index, and the left ventricular volume ratio, the automatically quantified value of the myocardial ischemia, the cardiac function information, and the phase information of the subject or the like is stored) is referred to as a model target.

The creation of the trained model 107a will be described. The trained model 107a is created by a learning model creation device. That is, the learning model creation device creates the trained model 107a. In addition, the diagnosis assistance device 100a may include the learning model creation device. That is, the diagnosis assistance device 100a may create the trained model 107a.

The learning model creation device 200a trains a learning model (a model that is the basis of the trained model 107a) using a learning dataset in which the combination of at least one of the index for predicting the onset of the coronary artery disease, the coronary artery calcium score, the body mass index, and the left ventricular volume ratio, the automatically quantified value of the myocardial ischemia, the cardiac function information, and the phase information of the subject of the model target or the like is used as an input sample and the result of the all-cause mortality of the subject is used as an output sample, thereby creating the trained model 107a. The input sample is data that is input to the input layer when the learning model is trained. The output sample is data (training data) that is the correct answer to be compared with the output value output from the output layer when the learning model is trained.

A learning dataset is input to the input unit 202. The reception unit 204 acquires the learning dataset from the input unit 202. The input sample and the output sample are paired and the learning dataset includes a plurality of pairs. The input sample may be some of at least one of the index for predicting the onset of the coronary artery disease, the coronary artery calcium score, the body mass index, and the left ventricular volume ratio, the automatically quantified value of the myocardial ischemia, the cardiac function information, the phase information, and the like (sometimes referred to as a “partial combination or the like”) instead of a combination of at least one of the index for predicting the onset of the coronary artery disease, the coronary artery calcium score, the body mass index, and the left ventricular volume ratio, the automatically quantified value of the myocardial ischemia, the cardiac function information, the phase information, and the like (sometimes referred to as a “total combination or the like”).

The output sample (the result of predicting the all-cause mortality) is acquired from the result of the all-cause mortality of the model target.

The processing unit 206a inputs an input sample to the input layer of the learning model 207a with respect to all pairs, calculates an error between the output value output from the output layer and the output sample (training data) corresponding to the input sample, and changes a parameter of the learning model 207a (trains the learning model 207a) so that the error is minimized, and creates a trained model 107a.

The trained model 107a created as described above is received by the diagnosis assistance device 100a from the learning model creation device 200a via a network or a medium and acquired by the processing unit 106a. When the learning model creation device 200a is included in the diagnosis assistance device 100a, the processing unit 106a acquires the trained model 107a from the learning model creation device 200a.

The acquisition of the result of predicting the all-cause mortality by the trained model 107a will be described. The processing unit 106a inputs a combination of at least one of the index for predicting the onset of the coronary artery disease, the coronary artery calcium score, the body mass index, and the left ventricular volume ratio, the automatically quantified value of the myocardial ischemia, the cardiac function information, and the phase information of the subject to the trained model 107a and obtains an output value from the trained model 107a. In addition, when the trained model 107a is created using a partial combination or the like as an input sample, the processing unit 106a inputs the partial combination or the like to the trained model 107a.)

A configuration in which the result of predicting the coronary artery disease is acquired by the trained model 107a may be adopted. In this case, the processing unit. 106a inputs the combination of at least one of the index for predicting the onset of the left ventricular volume ration, the automatically quantified value of the myocardial ischemia, the cardiac function information, and the phase information of the subject and the like to the trained model 107a in which the combination of at least one of the index for predicting the onset of the coronary artery disease, the coronary artery calcium score, the body mass index, and the left ventricular volume ratio, the automatically quantified value of the myocardial ischemia, the cardiac function information, and the phase information of the subject, who is a subject from which the result of the coronary artery disease has been actually acquired, and the like are stored to acquire the result of predicting the coronary artery disease of the subject.

Hereinafter, the combination of at least one of the index for predicting the onset of the coronary artery disease, the coronary artery calcium score, the body mass index, and the left ventricular volume ratio, the automatically quantified value of the myocardial ischemia, the cardiac function information, and the phase information related to the creation of the trained model 107a (a subject from whom the result of the coronary artery disease has been actually acquired as a subject whose combination of at least one of the index for predicting the onset of the coronary artery disease, the coronary artery calcium score, the body mass index, and the left ventricular volume ratio, the automatically quantified value of the myocardial ischemia, the cardiac function information, and the phase information of the subject or the like is stored) is referred to as a model target.

The creation of the trained model 107a will be described. The trained model 107a is created by a learning model creation device. That is, the learning model creation device creates the trained model 107a. In addition, the diagnosis assistance device 100a may include the learning model creation device. That is, the diagnosis assistance device 100a may create the trained model 107a.

The learning model creation device 200a trains a learning model (a model that is the basis of the trained model 107a) using a learning dataset in which the combination of at least one of the index for predicting the onset of the coronary artery disease, the coronary artery calcium score, the body mass index, and the left ventricular volume ratio, the automatically quantified value of the myocardial ischemia, the cardiac function information, and the phase information of the subject of the model target or the like is used as an input sample and the result of the coronary artery disease of the subject is used as an output sample, thereby creating the trained model 107a. The input sample is data that is input to the input layer when the learning model is trained. The output sample is data (training data) that is the correct answer to be compared with the output value output from the output layer when the learning model is trained.

A learning dataset is input to the input unit 202. The reception unit 204 acquires the learning dataset from the input unit 202. The input sample and the output sample are paired and the learning dataset includes a plurality of pairs. The input sample may be some of at least one of the index for predicting the onset of the coronary artery disease, the coronary artery calcium score, the body mass index, and the left ventricular volume ratio, the automatically quantified value of the myocardial ischemia, the cardiac function information, the phase information, and the like (sometimes referred to as a “partial combination or the like”) instead of a combination of at least one of the index for predicting the onset of the coronary artery disease, the coronary artery calcium score, the body mass index, and the left ventricular volume ratio, the automatically quantified value of the myocardial ischemia, the cardiac function information, the phase information, and the like (sometimes referred to as a “total combination or the like”).

The output sample (the result of predicting the coronary artery disease) is acquired from the result of the coronary artery disease of the model target.

The processing unit 206a inputs an input sample to the input layer of the learning model 207a with respect to all pairs, calculates an error between the output value output from the output layer and the output sample (training data) corresponding to the input sample, and changes a parameter of the learning model 207a (trains the learning model 207a) so that the error is minimized, and creates a trained model 107a.

The trained model 107a created as described above is received by the diagnosis assistance device 100a from the learning model creation device 200a via a network or a medium and acquired by the processing unit 106a. When the learning model creation device 200a is included in the diagnosis assistance device 100a, the processing unit 106a acquires the trained model 107a from the learning model creation device 200a.

A combination of at least one of the index for predicting the onset of the coronary artery disease, the coronary artery calcium score, the body mass index, and the left ventricular volume ratio, the automatically quantified value of the myocardial ischemia, the cardiac function information, and the phase information of the subject is input to the trained model 107a and an output value is obtained from the trained model 107a. In addition, when the trained model 107a is created using a partial combination or the like as an input sample, the processing unit 106a inputs the partial combination or the like to the trained model 107a.

According to the diagnosis assistance device according to Modified Example 1 of the embodiment, the diagnosis assistance device 100a receives at least one of the index for predicting the onset of the coronary artery disease acquired before the stress myocardial scintigraphy is performed on the subject, the coronary artery calcium score of the subject acquired when the stress myocardial scintigraphy is performed, the body mass index of the subject, and the left ventricular volume ratio during stress and rest of the subject, the automatically quantified value of the myocardial ischemia, the cardiac function information, and the phase information and acquires at least one of the predicted value of the result of performing the reperfusion therapy, the result of predicting the onset of the heart failure, the result of predicting the cardiac death, the result of predicting the all-cause mortality, and the result of predicting the coronary artery disease on the basis of at least one of the index for predicting the onset of the coronary artery disease, the coronary artery calcium score, the body mass index, and the left ventricular volume ratio, the automatically quantified value of the myocardial ischemia, the cardiac function information, and the phase information that have been received, and the trained model. Therefore, it is possible to acquire at least one of the predicted value of the result of performing the reperfusion therapy, the result of predicting the onset of the heart failure, the result of predicting the cardiac death, the result of predicting the all-cause mortality, and the result of predicting the coronary artery disease for the subject, without relying on human visual inspection.

According to the learning model creation device according to Modified Example 1 of the embodiment, the learning model creation device 200a receives a learning dataset in which at least one of the index for predicting the onset of the coronary artery disease acquired before the stress myocardial scintigraphy is performed on the subject, the coronary artery calcium score of the subject acquired when the stress myocardial scintigraphy is performed, the body mass index of the subject, and the left ventricular volume ratio during stress and rest of the subject, the automatically quantified value of the myocardial ischemia, the cardiac function information, and the phase information are included as learning data and creates a learning model by performing machine learning on a relationship of the automatically quantified value of the myocardial ischemia, the cardiac function information, the phase information, and at least one of the result of performing the reperfusion therapy, the result of the onset of the heart failure, the result of the cardiac death, the result of the all-cause mortality, and the result of the coronary artery disease using at least one of the index for predicting the onset of the coronary artery disease, the coronary artery calcium score, the body mass index, and the left ventricular volume ratio, the automatically quantified value of the myocardial ischemia, the cardiac function information, and the phase information as explanatory variables and using at least one of the result of performing the reperfusion therapy, the result of the onset of the heart failure, the result of the cardiac death, the result of the all-cause mortality, and the result of the coronary artery disease as an objective variable.

### Modified Example 2 of Embodiment

A diagnosis assistance device 100b according to Modified Example 2 of the embodiment will be described.

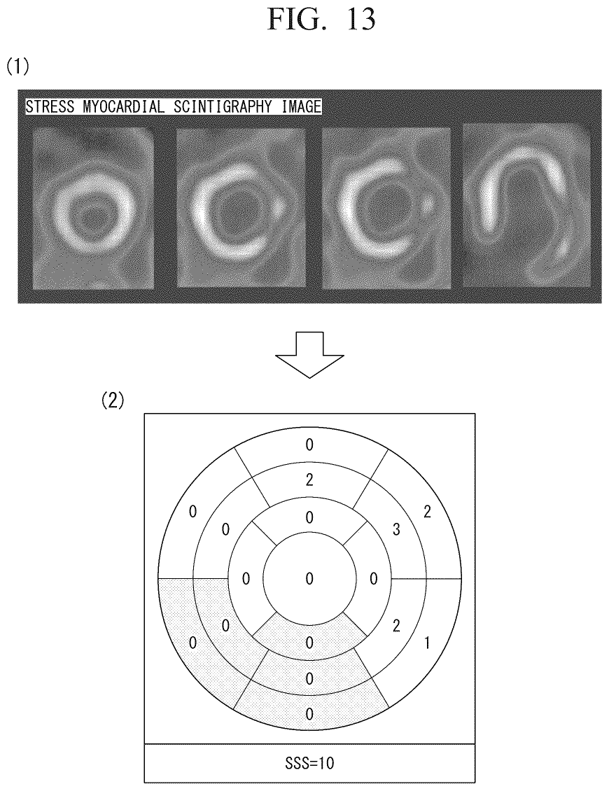

FIG. 12 is a diagram showing an example of the diagnosis assistance device according to Modified Example 2 of the embodiment.

Unlike the diagnosis assistance device 100 of the embodiment, the diagnosis assistance device 100b of Modified Example 2 of the embodiment receives a visual semi-quantitative index of the subject's image acquired by myocardial scintigraphy or subject-related information including an automatically quantified value of myocardial ischemia of the subject and the visual semi-quantitative index of the subject's image, instead of an automatically quantified value of myocardial ischemia of the subject. Hereinafter, a case where the subject-related information including the visual semi-quantitative index of the subject's image acquired by the myocardial scintigraphy is received instead of the automatically quantified value of myocardial ischemia of the subject will be described as an example. The present invention can also be applied to the case where the subject-related information including the automatically quantified value of the myocardial ischemia of the subject and the visual semi-quantitative index of the subject's image is received instead of the automatically quantified value of the myocardial ischemia of the subject.

The diagnosis assistance device 100b acquires at least one of a predicted value of a result of performing reperfusion therapy, a result of predicting the onset of heart failure, a result of predicting cardiac death, a result of predicting all-cause mortality, and a result of predicting coronary artery disease on the basis of cardiac function information, phase information, and the visual semi-quantitative index included in the received subject-related information and a trained model. Here, the trained model is obtained by performing machine learning on a relationship between a combination of the cardiac function information, the phase information, and the visual semi-quantitative index and at least one of the predicted value of the result of performing the reperfusion therapy, the result of predicting the onset of the heart failure, the result of predicting the cardiac death, the result of predicting the all-cause mortality, and the result of predicting the coronary artery disease.

The diagnosis assistance device 100b outputs subject identification information and at least one of the predicted value of the result of performing the reperfusion therapy, the result of predicting the onset of the heart failure, the result of predicting the cardiac death, the result of predicting the all-cause mortality, and the result of predicting the coronary artery disease that have been acquired.

The diagnosis assistance device 100b is implemented by a device such as a personal computer, a server, a smartphone, a tablet computer, or an industrial computer. The diagnosis assistance device 100b includes an input unit 102, a reception unit. 104, a processing unit 106b, an output unit 108, and a storage unit 110.

The reception unit 104 acquires the subject-related information from the input unit 102. The reception unit 104 acquires the subject identification information, the cardiac function information, the phase information, and the visual semi-quantitative index included in the acquired subject-related information and receives the subject identification information, the cardiac function information, the phase information, and the visual semi-quantitative index that have been acquired.

The visual semi-quantitative index is derived from visual assessment of myocardial scintigraphy images and a score assigned to each myocardial segment in accordance with a predefined scale.

FIG. 13 is an explanatory diagram of the visual semi-quantitative index. In FIG. 13. (1) is a diagram showing an example of a stress myocardial scintigraphy image, and (2) is a diagram showing an example of a myocardial segment. When the visual semi-quantitative index is derived, a score is given to each myocardial segment on the basis of the stress myocardial scintigraphy image. Specifically, a score of 0 is given for normal cases, a score of 1 is given for mild accumulation reduction, a score of 2 is given for mild abnormalities, a score of 3 is given for moderate abnormalities, and a score of 4 is given for no accumulation (severe abnormality). A summed rest score (SRS) is derived by calculating a sum of scores during rest. A summed stress score (SSS) is derived by calculating a sum of the scores during stress. A summed difference score (SDS) is derived by subtracting the SRS from the SSS.

The processing unit 106b acquires the subject identification information, the cardiac function information, the phase information, and the visual semi-quantitative index from the reception unit 104. The processing unit 106b includes a trained model 107b. The trained model 107b is obtained by performing machine learning on a relationship between a combination of the cardiac function information, the phase information, and the visual semi-quantitative index and at least one of the result of performing the reperfusion therapy, the result of the onset of the heart failure, the result of the cardiac death, the result of the all-cause mortality, and the result of the coronary artery disease. The processing unit 106b inputs the combination of the cardiac function information, the phase information, and the visual semi-quantitative index that have been acquired to the trained model 107b and acquires at least one of the predicted value of the result of performing the reperfusion therapy, the result of predicting the onset of the heart failure, the result of predicting the cardiac death, the result of predicting the all-cause mortality, and the result of predicting the coronary artery disease output by the trained model 107b with respect to the combination of the cardiac function information, the phase information, and the visual semi-quantitative index that have been input.

All or a part of the processing unit 106b is a functional unit (hereinafter referred to as a software functional unit) that is implemented by a processor such as a CPU executing a program stored in the storage unit 110. In addition, all or a part of the processing unit 106b may be implemented by hardware such as an ISI circuit, an ASIC, or an FPGA or may be implemented by a combination of a software functional unit and hardware.

### (Operation of Diagnosis Assistance Device 100b)

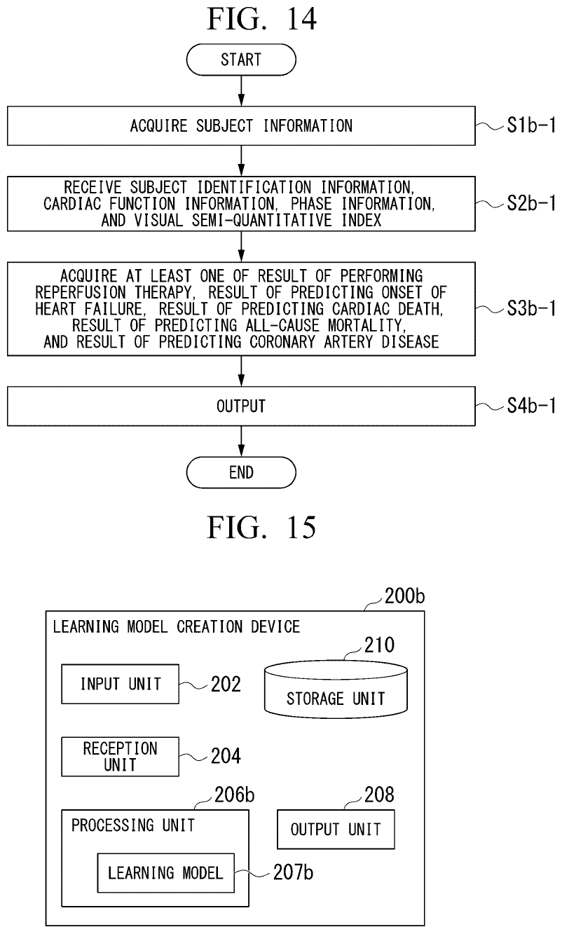

FIG. 14 is a flowchart showing an example of the operation of the diagnosis assistance device according to Modified Example 2 of the embodiment.

### (Step S1b-1)

The input unit 102 acquires subject-related information.

### (Step S2b-1)

The reception unit 104 acquires the subject-related information from the input unit 102. The reception unit 104 acquires the subject identification information, the cardiac function information, the phase information, and the visual semi-quantitative index included in the acquired subject-related information and receives the subject identification information, the cardiac function information, the phase information, and the visual semi-quantitative index that have been acquired.

### (Step S3b-1)

The processing unit 106b acquires the subject identification information, the cardiac function information, the phase information, and the visual semi-quantitative index from the reception unit 104. The processing unit 106b inputs a combination of the cardiac function information, the phase information, and the visual semi-quantitative index that have been acquired to the trained model 107b and acquires at least one of the predicted value of the result of performing the reperfusion therapy, the result of predicting the onset of the heart failure, the result of predicting the cardiac death, the result of predicting the all-cause mortality, and the result of predicting the coronary artery disease output by the trained model 107b with respect to the input combination of the cardiac function information, the phase information, and the visual semi-quantitative index.

### (Step S4b-1)

The output unit 108 acquires the subject identification information and at least one of the predicted value of the result of performing the reperfusion therapy, the result of predicting the onset of the heart failure, the result of predicting the cardiac death, the result of predicting the all-cause mortality, and the result of predicting the coronary artery disease from the processing unit 106b. The output unit 108 outputs the subject identification information and at least one of the predicted value of the result of performing the reperfusion therapy, the result of predicting the onset of the heart failure, the result of predicting the cardiac death, the result of predicting the all-cause mortality, and the result of predicting the coronary artery disease that have been acquired.

In Modified Example 2 of the embodiment described above, the processing unit 106b inputs the combination of the cardiac function information, the phase information, and the visual semi-quantitative index of the subject and the like to the trained model 107b in which the combination of the cardiac function information, the phase information, and the visual semi-quantitative index of the subject, who is a subject from which at least one of the result of performing the reperfusion therapy, the result of the onset of the heart failure, the result of predicting the cardiac death, the result of predicting the all-cause mortality, and the result of predicting the coronary artery disease has been actually acquired, or the like is stored to acquire at least one of the predicted value of the result of performing the reperfusion therapy on the subject, the result of predicting the onset of heart failure, the result of predicting the cardiac death, the result of predicting the all-cause mortality, and the result of predicting the coronary artery disease.

Hereinafter, the combination of the cardiac function information, the phase information, and the visual semi-quantitative index related to the creation of the trained model 107b (a subject from whom at least one of the result of performing the reperfusion therapy, the result of the onset of the heart failure, the result of predicting the cardiac death, the result of predicting the all-cause mortality, and the result of predicting the coronary artery disease has been actually acquired as a subject whose combination of the cardiac function information, the phase information, and the visual semi-quantitative index of the subject or the like is stored) is referred to as a model target.

The creation of the trained model 107b will be described. The trained model 107b is created by a learning model creation device. That is, the learning model creation device creates the trained model 107b. In addition, the diagnosis assistance device 100b may include the learning model creation device. That is, the diagnosis assistance device 100b may create the trained model 107b.

FIG. 15 is a diagram showing an example of a learning model creation device according to Modified Example 2 of the embodiment. The learning model creation device 200b according to Modified Example 2 of the embodiment is implemented by a device such as a personal computer, a server, a smartphone, tablet industrial computer.

The learning model creation device 200b trains a learning model (a model that is the basis of the trained model 107b) using a learning dataset in which the combination of the cardiac function information, the phase information, and the visual semi-quantitative index of the subject of the model target or the like is used as an input sample and at least one of the result of performing the reperfusion therapy on the subject, the result of the onset of the heart failure, the result of predicting the cardiac death, the result of predicting the all-cause mortality, and the result of predicting the coronary artery disease is used as an output sample, thereby creating the trained model 107b.

For example, the learning model creation device 200b uses algorithms of a CNN, an RNN, an LSTM, a random forest, an SVM, a neural network, and the like to construct the trained model 107b. The input sample is data to be input to an input layer when the learning model is trained. The output sample is data (training data) that is a correct answer to be compared with an output value output from an output layer when the learning model is trained.

The learning model creation device 200b includes an input unit 202, a reception unit 204, a processing unit 206b, an output unit 208, and a storage unit 210.

An input sample and an output sample are paired. The learning dataset includes a plurality of pairs. The input sample may be some of the cardiac function information, the phase information, the visual semi-quantitative index, and the like (sometimes referred to as a “partial combination or the like”) instead of a combination of the cardiac function information, the phase information, and the visual semi-quantitative index, and the like (sometimes referred to as a “total combination or the like”).

The output sample (at least one of the predicted value of the result of performing the reperfusion therapy, the result of predicting the onset of the heart failure, the result of predicting the cardiac death, the result of predicting the all-cause mortality, and the result of predicting the coronary artery disease) is acquired from at least one of the result of performing the reperfusion therapy, the result of the onset of the heart failure, the result of predicting the cardiac death, the result of predicting the all-cause mortality, and the result of predicting the coronary artery disease of the model target.

The learning model creation device 200b inputs an input sample to the input layer of the learning model 207b with respect to all pairs, calculates an error between the output value output from the output layer and the output sample (training data) corresponding to the input sample, and changes the parameters of the learning model 207b (trains the learning model 207b) so that the error is minimized, and creates the trained model 107b.

The trained model 107b created as described above is received by the diagnosis assistance device 100b from the output unit 208 via a network or a medium and acquired by the processing unit 106b. When the learning model creation device 200b is included in the diagnosis assistance device 100b, the processing unit 106b acquires the trained model 107b from the learning model creation device 2000.

The acquisition of at least one of the predicted value of the result of performing the reperfusion therapy, the result of predicting the onset of the heart failure, the result of predicting the cardiac death, the result of predicting the all-cause mortality, and the result of predicting the coronary artery disease by the trained model 107b will be described. The processing unit 106b inputs the combination of the cardiac function information, the phase information, and the visual semi-quantitative index of the subject to the trained model 107b and obtains an output value from the trained model 107b.

In addition, when the trained model 107b is created using a partial combination or the like as an input sample, the processing unit 106b inputs the partial combination or the like to the trained model 107b.

### (Operation of Learning Model Creation Device 200b)

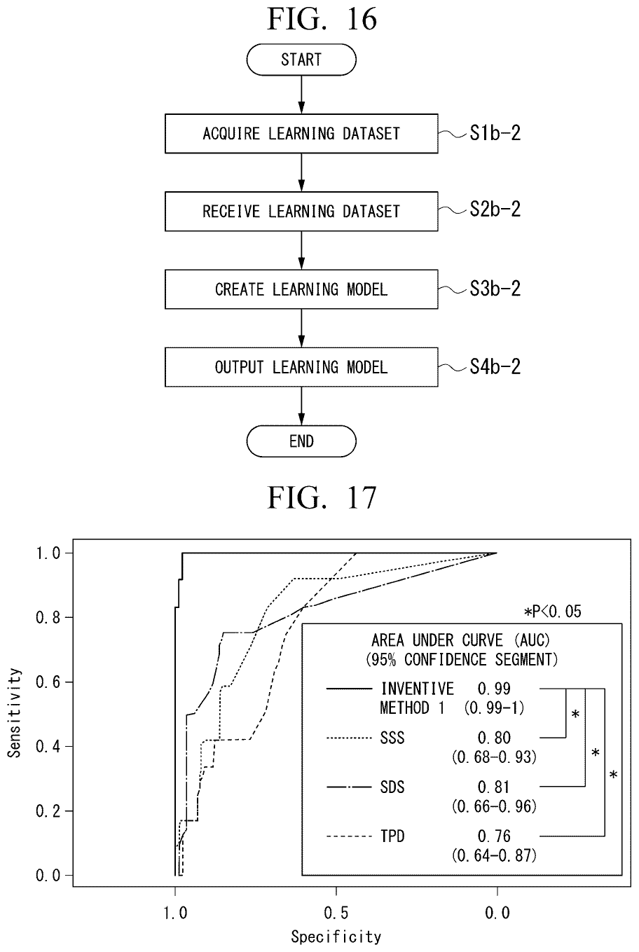

FIG. 16 is a flowchart showing an example of an operation of a learning model creation device according to Modified Example 2 of the embodiment.

### (Step S1b-2)

The input unit 202 acquires a learning dataset.

### (Step S2b-2)

The reception unit 204 acquires the learning dataset from the input unit 202. The reception unit 204 receives the acquired learning dataset.

### (Step S3b-2)

The processing unit 206b acquires the learning dataset from the reception unit 204. The processing unit 206b inputs the input sample to the input layer of the learning model 207b with respect to all pairs of input samples and output samples included in the learning dataset, calculates an error between the output value output from the output layer and the output sample (training data) corresponding to the input sample, changes the parameters of the learning model 207b (trains the learning model 207b) so that the error is minimized, and creates the trained model 107b.

### (Step S4b-2)

The output unit 208 acquires the learning model 207b from the processing unit 206b and outputs the acquired learning model 207b.

A configuration in which the result of performing the reperfusion therapy is acquired by the trained model 107b may be adopted. In this case, the processing unit 106b inputs the combination of the cardiac function information, the phase information, and the visual semi-quantitative index of the subject and the like to the trained model 107b in which the combination of the cardiac function information, the phase information, and the visual semi-quantitative index of the subject, who is a subject from whom the result of performing the reperfusion therapy has been actually acquired, and the like are stored to acquire the result of performing the reperfusion therapy on the subject.

Hereinafter, the combination of the cardiac function information, the phase information, and the visual semi-quantitative index related to the creation of the trained model 107b (the subject from whom the result of performing the reperfusion therapy has been actually acquired as the subject whose combination of the cardiac function information, the phase information, and the visual semi-quantitative index of the subject or the like is stored) is referred to as a model target.

The creation of the trained model 107b will be described. The trained model 107b is created by the learning model creation device 200b. That is, the learning model creation device 200b creates the trained model 107b. In addition, the diagnosis assistance device 100b may include the learning model creation device 2006. That is, the diagnosis assistance device 100b may create the trained model 107b.

The learning model creation device 200b trains a learning model 207b (a model that is the basis of the trained model 107b) using a learning dataset in which the combination of the cardiac function information, the phase information, and the visual semi-quantitative index of the subject of the model target or the like is used as an input sample and the result of performing the reperfusion therapy on the subject is used as an output sample, thereby creating the trained model 107b. The input sample is data that is input to the input layer when the learning model is trained. The output sample is data (training data) that is the correct answer to be compared with the output value output from the output layer when the learning model 207b is trained.

A learning dataset is input to the input unit 202. The reception unit 204 acquires the learning dataset from the input unit 202. An input sample and an output sample are paired and the learning dataset includes a plurality of pairs. The input sample may be some of the cardiac function information, the phase information, the visual semi-quantitative index, and the like (sometimes referred to as a “partial combination or the like”) instead of a combination of the cardiac function information, the phase information, the visual semi-quantitative index, and the like (sometimes referred to as a “total combination or the like”).

The output sample (the result of performing the reperfusion therapy) is acquired from the result of performing the reperfusion therapy on the model target.

The learning model creation device 200b inputs an input sample to the input layer of the learning model 207b with respect to all pairs, calculates an error between the output value output from the output layer and the output sample (training data) corresponding to the input sample, changes a parameter of the learning model 207b (trains the learning model) so that the error is minimized, and creates the trained model 107b.

The trained model 107b created as described above is received by the diagnosis assistance device 100b from the learning model creation device 200b via a network or a medium and acquired by the processing unit 106b. When the learning model creation device 200b is included in the diagnosis assistance device 100b, the processing unit 106b acquires the trained model 107b from the learning model creation device 200b.

The acquisition of the result of performing the reperfusion therapy by the trained model 107b will be described. The processing unit 106b inputs the combination of the cardiac function information, the phase information, the visual semi-quantitative index of the subject to the trained model 107b and obtains an output value from the trained model 107b. In addition, when the trained model 107b is created using a partial combination or the like ax an input sample, the processing unit 106b inputs the partial combination or the like to the trained model 107b.

A configuration in which the result of predicting the onset of the heart failure is acquired by the trained model 107b may be adopted. In this case, the processing unit 106b inputs the combination of the cardiac function information, the phase information, and the visual semi-quantitative index of the subject and the like to the trained model 107b in which the combination of the cardiac function information, the phase information, and the visual semi-quantitative index of the subject, who is a subject from whom the result of the onset of the heart failure has been actually acquired, and the like are stored to acquire the result of predicting the onset of the heart failure of the subject.

Hereinafter, the combination of the cardiac function information, the phase information, and the visual semi-quantitative index related to the creation of the trained model 107b (the subject from whom the result of the onset of the heart failure has been actually acquired as the subject whose combination of the cardiac function information, the phase information, and the visual semi-quantitative index of the subject or the like is stored) is referred to as a model target.

The creation of the trained model 107b will be described. The trained model 107b is created by the learning model creation device 200b. That is, the learning model creation device 200b creates the trained model 107b. In addition, the diagnosis assistance device 100b may include the learning model creation device 200b. That is, the diagnosis assistance device 100b may create the trained model 107b.

The learning model creation device 200b trains a learning model 207b (a model that is the basis of the trained model 107b) using a learning dataset in which the combination of the cardiac function information, the phase information, and the visual semi-quantitative index of the subject of the model target or the like is used as an input sample and the result of the onset of the heart failure of the subject is used as an output sample, thereby creating the trained model 107b. The input sample is data that is input to the input layer when the learning model 207b is trained. The output sample is data (training data) that is the correct answer to be compared with the output value output from the output layer when the learning model 207b is trained.

A learning dataset is input to the input unit 202. The reception unit 204 acquires the learning dataset from the input unit 202. An input sample and an output sample are paired and the learning dataset includes a plurality of pairs. The input sample may be some of the cardiac function information, the phase information, the visual semi-quantitative index, and the like (sometimes referred to as a “partial combination or the like”) instead of a combination of the cardiac function information, the phase information, the visual semi-quantitative index, and the like (sometimes referred to as a “total combination or the like”).

The output sample (the result of predicting the onset of the heart failure) is acquired from the result of the onset of the heart failure of the model target.

The processing unit 206b inputs an input sample to the input layer of the learning model 207b with respect to all pairs, calculates an error between the output value output from the output layer and the output sample (training data) corresponding to the input sample, changes a parameter of the learning model 207b (trains the learning model) so that the error is minimized, and creates the trained model 107b.

The trained model 107b created as described above is received by the diagnosis assistance device 100b from the learning model creation device 200b via a network or a medium and acquired by the processing unit 106b. When the model creation device is included in the diagnosis assistance device 100b, the processing unit 106b acquires the trained model 107b from the model creation device.

The acquisition of the result of predicting the onset of the heart failure by the trained model 107b will be described. The processing unit 106b inputs the combination of the cardiac function information, the phase information, and the visual semi-quantitative index of the subject to the trained model 107b and obtains an output value from the trained model 107b. In addition, when the trained model 107b is created using a partial combination or the like as an input sample, the processing unit 106b inputs the partial combination or the like to the trained model 107b.

A configuration in which the result of predicting the cardiac death is acquired by the trained model 107b may be adopted. In this case, the processing unit 106b inputs the combination of the cardiac function information, the phase information, and the visual semi-quantitative index of the subject and the like to the trained model 107b in which the combination of the cardiac function information, the phase information, and the visual semi-quantitative index of the subject, who is a subject from whom the result of the cardiac death has been actually acquired, and the like are stored to acquire the result of predicting the cardiac death of the subject.

Hereinafter, the combination of the cardiac function information, the phase information, and the visual semi-quantitative index related to the creation of the trained model 107b (the subject from whom the result of the cardiac death has been actually acquired as the subject whose combination of the cardiac function information, the phase information, and the visual semi-quantitative index of the subject or the like is stored) is referred to as a model target.

The creation of the trained model 107b will be described. The trained model 107b is created by the learning model creation device 200b. That is, the learning model creation device 200b creates the trained model 107b. In addition, the diagnosis assistance device 100b may include the learning model creation device 200b. That is, the diagnosis assistance device 100b may create the trained model 107b.

The learning model creation device 200b trains a learning model 207b (a model that is the basis of the trained model 107b) using a learning dataset in which the combination of the cardiac function information, the phase information, and the visual semi-quantitative index of the subject of the model target or the like is used as an input sample and the result of the cardiac death of the subject is used as an output sample, thereby creating the trained model 107b. The input sample is data that is input to the input layer when the learning model 207b is trained. The output sample is data (training data) that is the correct answer to be compared with the output value output from the output layer when the learning model 207b is trained.

A learning dataset is input to the input unit 202. The reception unit 204 acquires the learning dataset from the input unit 202. An input sample and an output sample are paired and the learning dataset includes a plurality of pairs. The input sample may be some of the cardiac function information, the phase information, the visual semi-quantitative index, and the like (sometimes referred to as a “partial combination or the like”) instead of a combination of the cardiac function information, the phase information, the visual semi-quantitative index, and the like (sometimes referred to as a “total combination of the like”).

The output sample (the result of predicting the cardiac death) is acquired from the result of the cardiac death of the model target.

The processing unit 206b inputs an input sample to the input layer of the learning model 207b with respect to all pairs, calculates an error between the output value output from the output layer and the output sample (training data) corresponding to the input sample, changes a parameter of the learning model 207b (trains the learning model) so that the error is minimized, and creates the trained model 107b.

The trained model 107b created as described above is received by the diagnosis assistance device 100b from the learning model creation device 200b via a network or a medium and acquired by the processing unit 106b. When the model creation device is included in the diagnosis assistance device 100b, the processing unit 106b acquires the trained model 107b from the model creation device.

The acquisition of the result of predicting the cardiac death by the trained model 107b will be described. The processing unit 106b inputs the combination of the cardiac function information, the phase information, and the visual semi-quantitative index of the subject to the trained model 107b and obtains an output value from the trained model 107b. In addition, when the trained model 107b is created using a partial combination or the like as an input sample, the processing unit 106b inputs the partial combination of the like to the trained model 107b.

A configuration in which the result of predicting the all-cause mortality is acquired by the trained model 107b may be adopted. In this case, the processing unit 106b inputs the combination of the cardiac function information, the phase information, and the visual semi-quantitative index of the subject and the like to the trained model 107b in which the combination of the cardiac function information, the phase information, and the visual semi-quantitative index of the subject, who is a subject from whom the result of the all-cause mortality has been actually acquired, and the like are stored to acquire the result of predicting the all-cause mortality of the subject.

Hereinafter, the combination of the cardiac function information, the phase information, and the visual semi-quantitative index related to the creation of the trained model 107b (the subject from whom the result of the all-cause mortality has been actually acquired as the subject whose combination of the cardiac function information, the phase information, and the visual semi-quantitative index of the subject or the like is stored) is referred to as a model target.

The creation of the trained model 107b will be described. The trained model 107b is created by the learning model creation device 200b. That is, the learning model creation device 200b creates the trained model 107b. In addition, the diagnosis assistance device 100b may include the learning model creation device 200b. That is, the diagnosis assistance device 100b may create the trained model 107b.

The learning model creation device 200b trains a learning model 207b (a model that is the basis of the trained model 107b) using a learning dataset in which the combination of the cardiac function information, the phase information, and the visual semi-quantitative index of the subject of the model target or the like is used as an input sample and the result of the all-cause mortality of the subject is used as an output sample, thereby creating the trained model 107b. The input sample is data that is input to the input layer when the learning model 207b is trained. The output sample is data (training data) that is the correct answer to be compared with the output value output from the output layer when the learning model 207b is trained.

A learning dataset is input to the input unit 202. The reception unit 204 acquires the learning dataset from the input unit 202. An input sample and an output sample are paired and the learning dataset includes a plurality of pairs. The input sample may be some of the cardiac function information, the phase information, the visual semi-quantitative index, and the like (sometimes referred to as a “partial combination or the like”) instead of a combination of the cardiac function information, the phase information, the visual semi-quantitative index, and the like (sometimes referred to as a “total combination or the like”).

The output sample (the result of predicting the all-cause mortality) is acquired from the result of the all-cause mortality of the model target.)

The processing unit 206b inputs an input sample to the input layer of the learning model 207b with respect to all pairs, calculates an error between the output value output from the output layer and the output sample (training data) corresponding to the input sample, changes a parameter of the learning model 207b (trains the learning model) so that the error is minimized, and creates the trained model 107b.

The trained model 107b created as described above is received by the diagnosis assistance device 100b from the learning model creation device 200b via a network or a medium and acquired by the processing unit 106b. When the model creation device is included in the diagnosis assistance device 100b, the processing unit. 106b acquires the trained model 107b from the model creation device.

The acquisition of the result of predicting the all-cause mortality by the trained model 107b will be described. The processing unit 106b inputs the combination of the cardiac function information, the phase information, and the visual semi-quantitative index of the subject to the trained model 107b and obtains an output value from the trained model 107b. In addition, when the trained model 107b is created using a partial combination or the like as an input sample, the processing unit 106b inputs the partial combination or the like to the trained model 107b.

A configuration in which the result of predicting the coronary artery disease is acquired by the trained model 107b may be adopted. In this case, the processing unit 106b inputs the combination of the cardiac function information, the phase information, and the visual semi-quantitative index of the subject and the like to the trained model 107b in which the combination of the cardiac function information, the phase information, and the visual semi-quantitative index of the subject, who is a subject from whom the result of the coronary artery disease has been actually acquired, and the like are stored to acquire the result of predicting the coronary artery disease of the subject.

Hereinafter, the combination of the cardiac function information, the phase information, and the visual semi-quantitative index related to the creation of the trained model 107b (the subject from whom the result of the coronary artery disease has been actually acquired as the subject whose combination of the cardiac function information, the phase information, and the visual semi-quantitative index of the subject or the like is stored) is referred to as a model target.

The creation of the trained model 107b will be described. The trained model 107b is created by the learning model creation device 200b. That is, the learning model creation device 200b creates the trained model 107b. In addition, the diagnosis assistance device 100b may include the learning model creation device 200b. That is, the diagnosis assistance device 100b may create the trained model 107b.

The learning model creation device 200b trains a learning model 207b (a model that is the basis of the trained model 107b) using a learning dataset in which the combination of the cardiac function information, the phase information, and the visual semi-quantitative index of the subject of the model target or the like is used as an input sample and the result of the coronary artery disease of the subject is used as an output sample, thereby creating the trained model 107b. The input sample is data that is input to the input layer when the learning model 207b is trained. The output sample is data (training data) that is the correct answer to be compared with the output value output from the output layer when the learning model 207b is trained.

A learning dataset is input to the input unit 202. The reception unit 204 acquires the learning dataset from the input unit 202. An input sample and an output sample are paired and the learning dataset includes a plurality of pairs. The input sample may be some of the cardiac function information, the phase information, the visual semi-quantitative index, and the like (sometimes referred to as a “partial combination or the like”) instead of a combination of the cardiac function information, the phase information, the visual semi-quantitative index, and the like (sometimes referred to as a “total combination or the like”).

The output sample (the result of predicting the coronary artery disease) is acquired from the result of the coronary artery disease of the model target.

The processing unit 206b inputs an input sample to the input layer of the learning model 207b with respect to all pairs, calculates an error between the output value output from the output layer and the output sample (training data) corresponding to the input sample, changes a parameter of the learning model 207b (trains the learning model) so that the error is minimized, and creates the trained model 107b.

The trained model 107b created as described above is received by the diagnosis assistance device 100b from the learning model creation device 200b via a network or a medium and acquired by the processing unit 106b. When the model creation device is included in the diagnosis assistance device 100b, the processing unit 106b acquires the trained model 107b from the model creation device.

The acquisition of the result of predicting the coronary artery disease by the trained model 107b will be described. The processing unit 106b inputs the combination of the cardiac function information, the phase information, and the visual semi-quantitative index of the subject to the trained model 107b and obtains an output value from the trained model 107b. In addition, when the trained model 107b is created using a partial combination or the like as an input sample, the processing unit 106b inputs the partial combination or the like to the trained model 107b.

As described above, it is also possible to create the trained model 107b in which the combination of the cardiac function information, the phase information, the automatically quantified value of the myocardial ischemia, and the visual semi-quantitative index of the subject, who is a subject from whom at least one of the result of performing the reperfusion therapy, the result of the onset of the heart failure, the result of predicting the cardiac death, the result of predicting the all-cause mortality, and the result of predicting the coronary artery disease has been actually acquired, or the like is stored.

According to the diagnosis assistance device according to Modified Example 2 of the present embodiment, the diagnosis assistance device 100b can receive the visual semi-quantitative index of the subject and the cardiac function information and the phase information acquired when the stress myocardial scintigraphy is performed on the subject and can acquire at least one of the predicted value of the result of performing the reperfusion therapy, the result of predicting the onset of the heart failure, the result of predicting the cardiac death, the result of predicting the all-cause mortality, and the result of predicting the coronary artery disease on the basis of the visual semi-quantitative index, the cardiac function information, and the phase information that have been received, and a trained model. Therefore, it is possible to acquire at least one of the predicted value of the result of performing the reperfusion therapy, the result of predicting the onset of the heart failure, the result of predicting the cardiac death, the result of predicting the all-cause mortality, and the result of predicting the coronary artery disease for the subject, without relying on human visual inspection.

According to the learning model creation device according to Modified Example 2 of the embodiment, the learning model creation device 200b can receive a learning dataset in which the visual semi-quantitative index of the subject and the cardiac function information and the phase information acquired when the stress myocardial scintigraphy is performed on the subject are included as learning data and at least one of the result of performing the reperfusion therapy on the subject, the result of the onset of the heart failure, the result of the cardiac death, the result of the all-cause mortality, and the result of the coronary artery disease is included as training data and can create the learning model by performing machine learning on a relationship of the visual semi-quantitative index, the cardiac function information, the phase information, and at least one of the result of performing the reperfusion therapy, the result of the onset of the heart failure, the result of the cardiac death, the result of the all-cause mortality, and the result of the coronary artery disease using the visual semi-quantitative index, the cardiac function information, and the phase information as explanatory variables and using at least one of the result of performing the reperfusion therapy, the result of the onset of the heart failure, the result of the cardiac death, the result of the all-cause mortality, and the result of the coronary artery disease as an objective variable.

An example of the effect of the above-described diagnosis assistance device will be described. A receiver operating characteristic (ROC) was calculated for a pilot test in which an SVM was applied to create a trained model for 339 subjects.

The trained model used here is obtained by performing machine learning on a relationship of a combination of blood flow distribution information (TPDs during stress and rest), cardiac function information (left ventricular ejection fractions during stress and rest), left ventricular contraction phase information (phase information (BW, SD, and entropy during stress)), an automatically quantified value of myocardial ischemia, left ventricular volumes (during stress and rest), a Suita score, a body mass index, a visual semi-quantitative index, and a TID ratio, the result of performing the reperfusion therapy, and the result of predicting the onset of the heart failure.

FIG. 17 is a diagram showing an example of a comparison result of the receiver operating characteristics of the diagnosis assistance device according to the embodiment. In FIG. 17, the horizontal axis represents specificity and the vertical axis represents sensitivity. The specificity is a rate at which a negative subject is correctly identified as negative and the sensitivity is a rate at which a positive subject is correctly identified as positive. If the test is effective, this curve moves away from a line of 45 degrees to the upper left. The further away they are, the more effective the test is. FIG. 17 shows the occurrence prediction diagnosis results of ischemia-reperfusion therapy for Inventive Method 1 (the diagnosis assistance device 100b), semi-quantitative scoring (SSS or SDS) by an expert, and TPD.

In FIG. 17, an area under an ROC curve (an area under curve (AUC) between 0.0 and 1.0 on the vertical axis was calculated. According to FIG. 17, the AUC of Inventive Method 1 was 0.99, the AUC of SSS was 0.80, the AUC of SDS was 0.81, and the AUC of the TPD was 0.76. Inventive Method 1 was compared to SSS, SDS, and TPD and was significantly superior to SSS, SDS, and TPD.

In the diagnosis assistance device 100a and the diagnosis assistance device 100b, the comparison with SSS, SDS, and TPD was also compared and a significantly superior result was obtained.

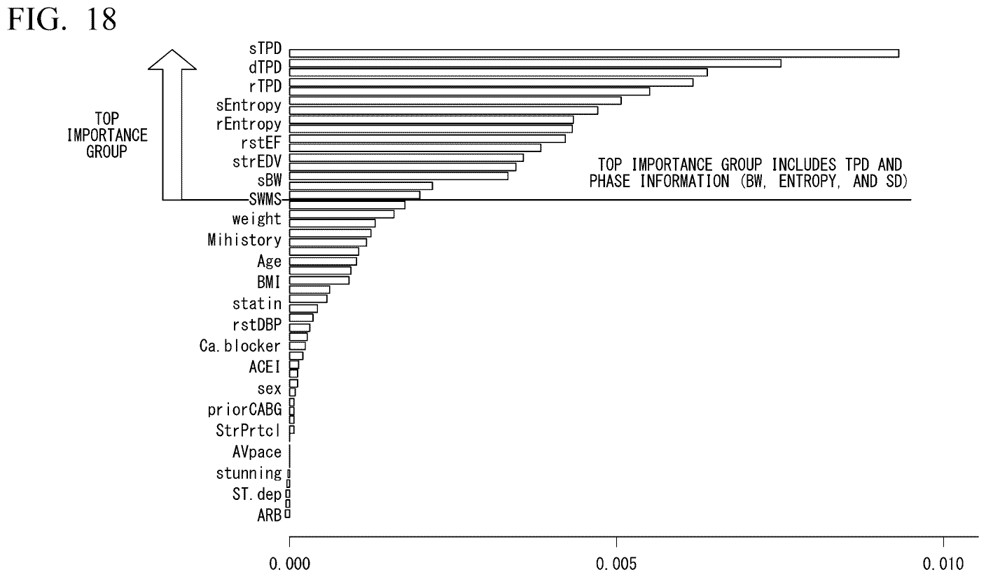

FIG. 18 is a diagram showing the importance of learning data used when a trained model is created according to the embodiment. FIG. 18 shows a result of analyzing the ranking of the importance of input information (learning data) when a predicted value of the result of performing the reperfusion therapy is output using a random forest of machine learning. As learning data, 49 items were considered. According to FIG. 18, the learning data are ranked in descending order of importance as follows: TPD (during stress), TPD (during stress-rest), TPD (during rest), phase information (SD) during rest), phase information (Entropy during stress), cardiac function information (left ventricular ejection fraction: EF during stress), phase information (Entropy during rest), left ventricular volume (left ventricular end-diastolic volume: EDV during rest), phase information (BW during rest), and phase information (BW during stress). In other words, TPD and phase information (BW. Entropy, and SD) are included in the top order of importance.

Moreover, the receiver operating characteristics were obtained for 1,200 subjects when deep learning was applied to create a trained model. The trained model used here was obtained by performing machine learning on a relationship between a combination of TPD, phase information, left ventricular ejection fraction, left ventricular volume, age, pre-examination blood pressure, BMI, medical history, sex, and oral medications and a result of diagnosing a multivessel coronary artery lesion (including a left main trunk, lesion).

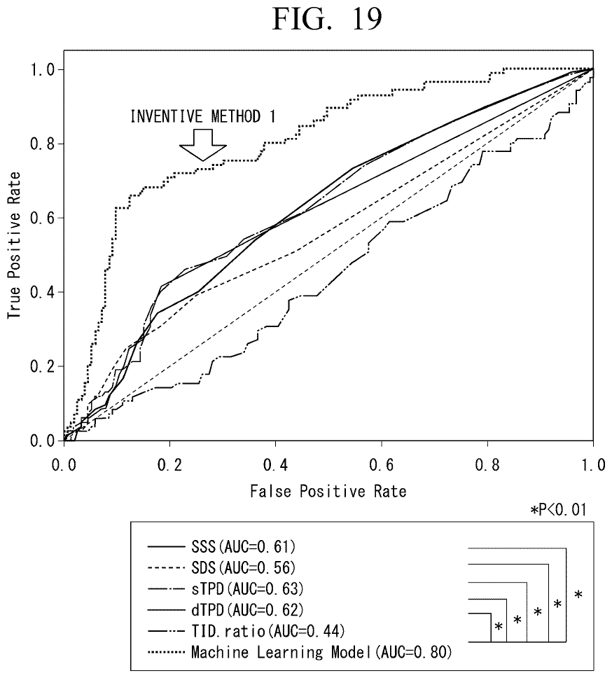

FIG. 19 is a diagram showing another example of the comparison result of the receiver operating characteristics of the diagnosis assistance device according to the embodiment. In FIG. 19, the horizontal axis represents a false positive rate and the vertical axis represents a true positive rate. Specificity is a rate at which a negative subject is correctly identified as negative. In FIG. 19, the occurrence prediction diagnosis results of ischemia-reperfusion therapy are shown for Inventive Method 1 (the diagnosis assistance device 100b), semi-quantitative scoring (SSS and SDS) by an expert, stress TPD (sTPD), stress-rest TPD (dTPD), and a TID ratio.

In FIG. 19, the area under the ROC curve (an area under curve (AUC) between 0.0 and 1.0 on the vertical axis was calculated. According to FIG. 19, the AUC of Inventive Method 1 was 0.80, the AUC of SSS was 0.61, the AUC of SDS was 0.56, STPD was 0.63, dTPD wax 0.62, and the TID ratio was 0.44. The result of Inventive Method 1 was significantly higher than those of SSS, SDS, sTPD, dTPD, and the TID ratio.

In the diagnosis assistance device 100a and the diagnosis assistance device 100b, the results were significantly superior to all of SSS, SDS, sTPD, dTPD, and the TID ratio,

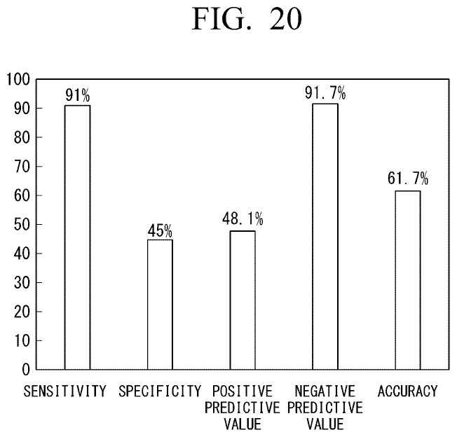

FIG. 20 is a diagram showing an example of the diagnostic capability of the diagnosis assistance device according to the embodiment. FIG. 20 shows a breakdown of the diagnostic capability of multivessel coronary artery lesions (including left main trunk lesions). In order to obtain the diagnostic capability with priority given to sensitivity required for a diagnosis assistance system, the following improvements were made. Weighting was applied to the learning data. Specifically, weighting was applied to abnormal cases. A dropout layer was added to prevent overlearning, and the amount of learning data was appropriately reduced. An elu function, which is an activation function, was introduced to prevent the disappearance of feature quantity information due to repeated calculations. An output threshold of deep learning was set from 0.5 to 0.3, making it easier to obtain a sensitivity-prioritized output. As a result, the results shown in FIG. 20 were obtained.

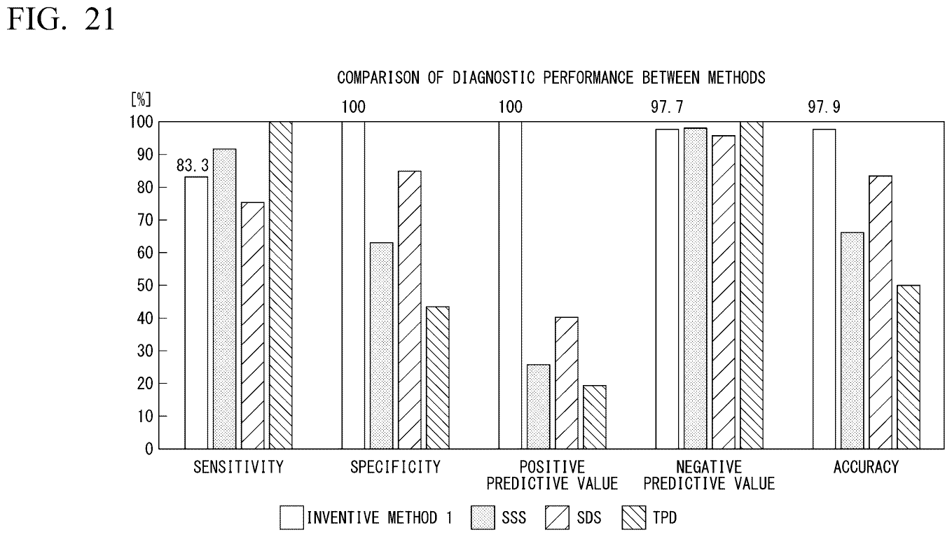

FIG. 21 is a diagram showing an example of a comparison result of the diagnostic capability of the diagnosis assistance device according to the embodiment. FIG. 21 shows the sensitivity, specificity, positive predictive value, and accuracy for each of Inventive Method 1 (the diagnosis assistance device 100b), semi-quantitative scoring (SSS and SDS) by an expert, and TPD. According to FIG. 21, Inventive Method 1 had a sensitivity of 83.3%, a specificity of 100%, a positive predictive value of 97.7%, and an accuracy of 97.9%, Inventive Method 1 showed significantly higher values in specificity, positive predictive value, and accuracy than SSS. SDS, and TPD, while the negative neutrality was also comparable at 97.7%.

The diagnosis assistance device 100 and the diagnosis assistance device 100a also showed significantly higher values in specificity, positive predictive value, and accuracy than the SSS, SDS, and TPD, while the negative neutral results were also comparable.

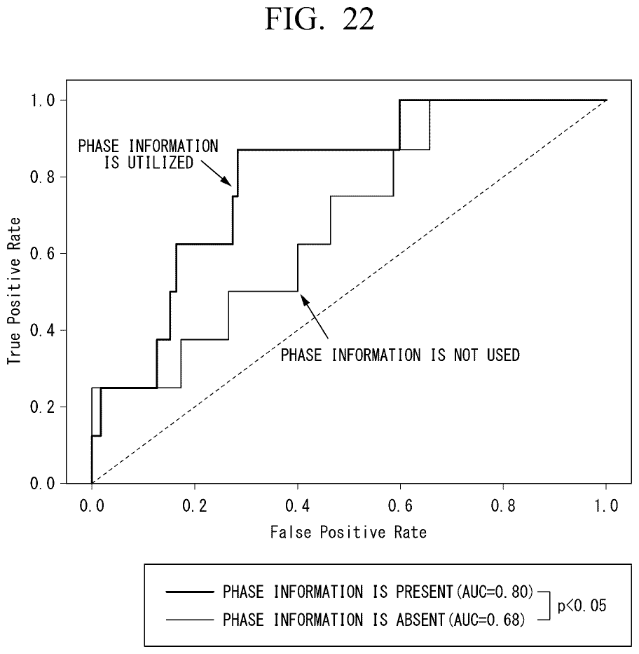

FIG. 22 is a diagram showing another example of the comparison result of the receiver operating characteristics of the diagnosis assistance device according to the embodiment. In FIG. 22, the horizontal axis represents a false positive rate and the vertical axis represents a true positive rate. The specificity is a rate at which a negative subject is correctly identified as negative. FIG. 22 shows the diagnostic capability of multivessel coronary artery lesions in Inventive Method 1 (the diagnosis assistance device 100b) when phase information is used (utilized) in the trained model and when phase information is not used (unused). The phase information is the bandwidth (BW), standard deviation (SD), and entropy during rest and stress.

In FIG. 22, the AUC was calculated. According to FIG. 22, the AUC when phase information was used was 0.80, and the AUC when phase information was not used was 0.68. It was shown that the trained model including phase information had significantly improved diagnostic capability compared to the model not including the 10 trained model. Similar results were obtained with the diagnosis assistance device 100a.

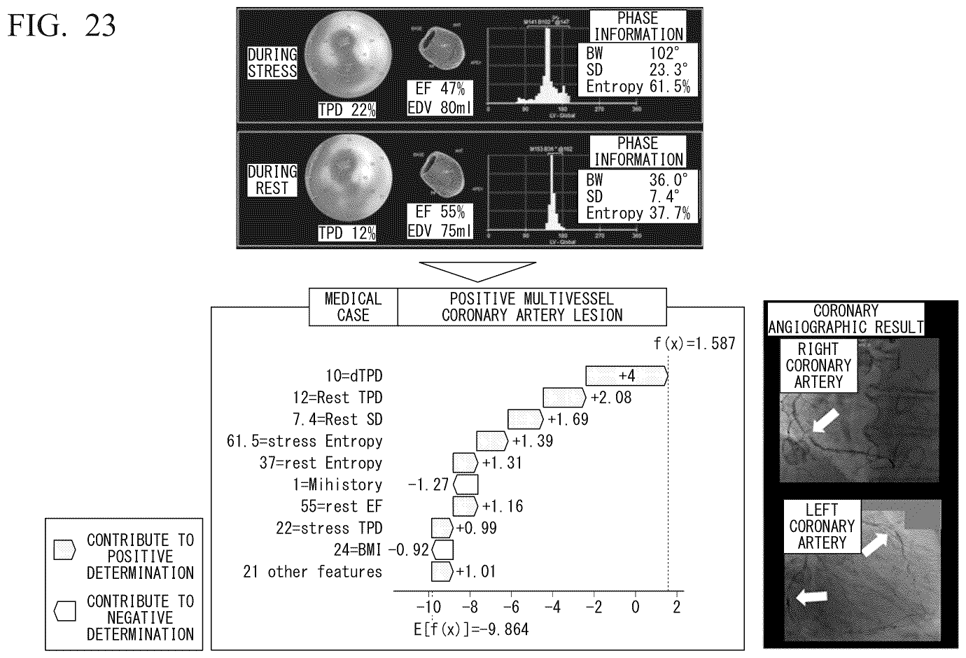

FIG. 23 is a diagram showing an example of implementation of explainability. Explainability (XAI) is a technique that enables humans to understand a process leading to prediction and inference results of artificial intelligence (AI).

In FIG. 23, the upper diagram shows information about the subject. The subject is a man in 80s with a BMI of 24 kg/m², a TID ratio of 1.07, and a history of myocardial infarction. Moreover, during stress, TPD 22%, EF 47%, EDV 80 ml, BW 102°, SD 23.3°, and Entropy 61.5% were measured. During rest, TPD 12%, EF 55%, EDV 75 ml, BW 36.0°, SD 7.4°, and Entropy 37.7% were measured.

In FIG. 23, the lower diagram shows the diagnostic basis and the coronary angiography results. Feature quantities including the above clinical information, TPD, EF, EDV, and phase information were input to the diagnosis assistance device, and diagnosis assistance for a positive multivessel coronary artery lesion was performed. Here, as an example, a display example of the diagnostic basis using SHapley Additive explanations (SHAP) values is shown.

According to the lower diagram, the results of dTPD, rest TPD, rest SD, stress Entropy, and rest Entropy were obtained in descending order on the basis of a degree of influence contributing to the determination of the positive multivessel coronary artery lesion.

In the coronary angiography, the arrows indicate the locations of the stenotic lesions. Lesions were found in three locations and the subject was diagnosed with a multivessel coronary artery lesion.

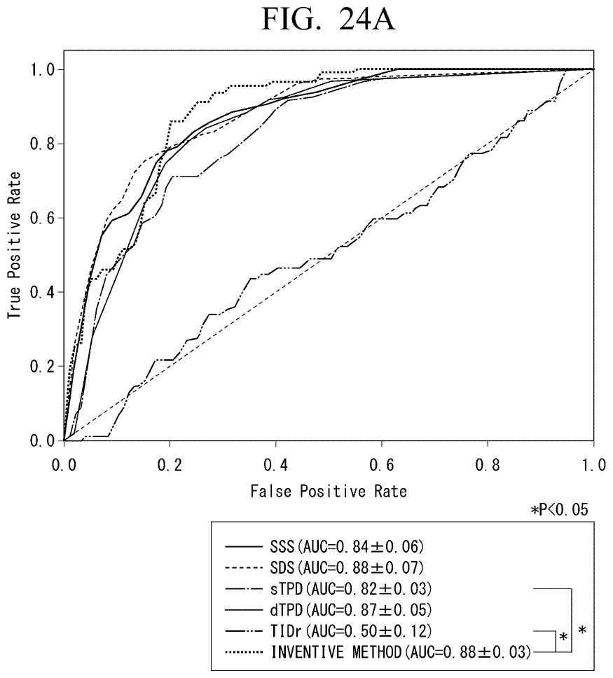

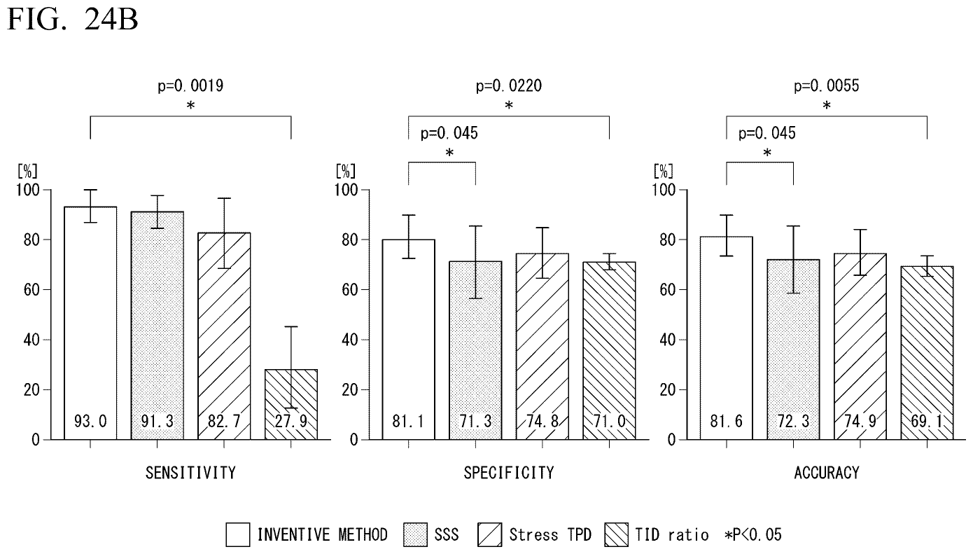

FIG. 24A is a diagram showing an example of a diagnosis result of the diagnosis assistance device of the embodiment. FIG. 24A shows an example of a diagnosis result of a multivessel lesion. In FIG. 24A, the horizontal axis represents a false positive rate and the vertical axis represents a true positive rate. In FIG. 24A, the diagnostic capability of the diagnosis assistance device 100 for a multivessel coronary artery lesion derived on the basis of the result of five random sampling trials for SSS and SDS by an expert, sTPD, dTPD, and TIDr (TID ratio), and the diagnosis assistance device is shown.

The AUC was calculated with respect to SSS. SDS, sTPD, dTPD, TIDr, and the case of the diagnosis assistance device 100b of the embodiment. The AUC for SSS was 0.84, the AUC for SDS was 0.88, the AUC for sTPD was 0.82, the AUC for dTPD was 0.87, the AUC for TIDr was 0.50, and the AUC for the diagnosis assistance device 100b of the embodiment was 0.88. It was shown that the diagnosis assistance device 100b of the present embodiment has significantly improved diagnostic capability compared to the conventional TIDr. Similar results were obtained with the diagnosis assistance device 100%.

FIG. 24B shows results of comparing sensitivity, specificity, and accuracy with respect to SSS, sTPD, TIDr, and the case of the diagnosis assistance device 100b of the embodiment. In FIG. 24B, the vertical axis represents a proportion where P<0.05. P indicates a significant difference. According to FIG. 24B, it can be seen that the diagnosis assistance device 100b of the present embodiment has improved not only the sensitivity but also the specificity and accuracy.

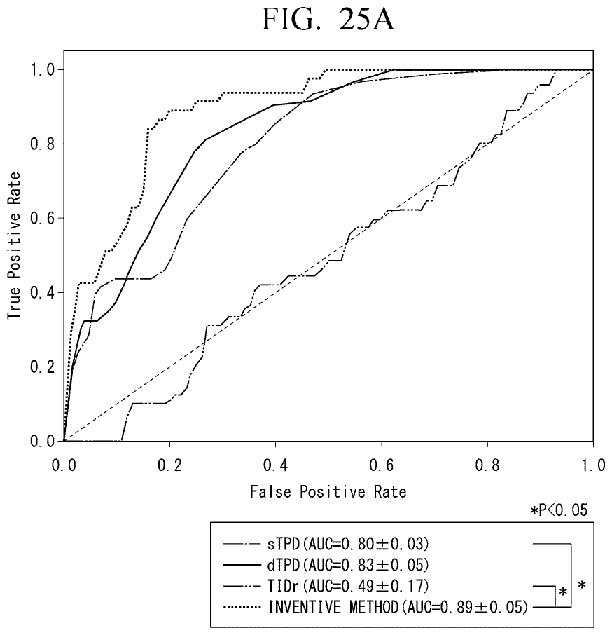

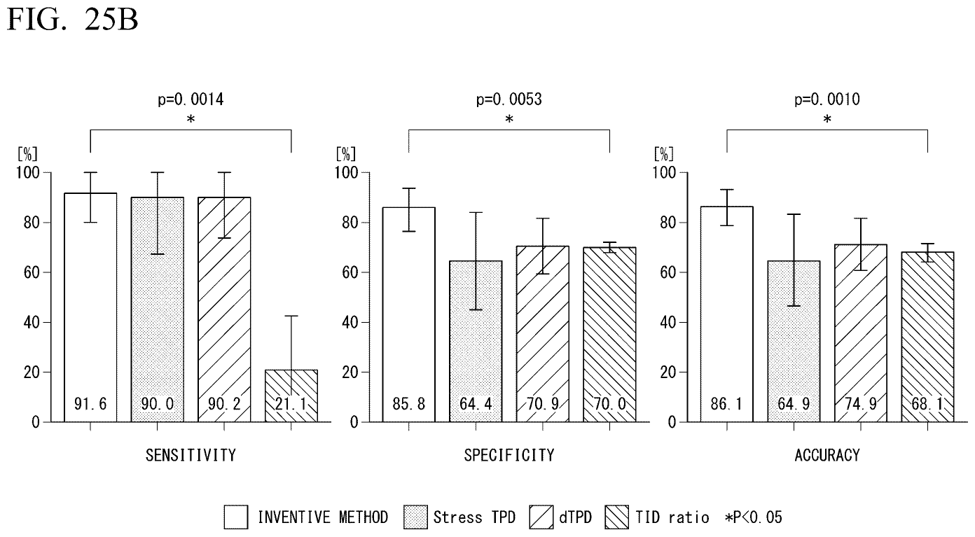

FIG. 25A is a diagram showing an example of a diagnosis result of the diagnosis assistance device of the embodiment. FIG. 25A shows an example of a diagnosis result of a multivessel lesion for a subject with normal or mild imaging findings (SSS<9). In FIG. 25A, the horizontal axis represents a false positive rate and the vertical axis represents a true positive rate. In FIG. 25A, in the diagnosis assistance device 100b, the diagnostic capability of a multivessel coronary artery lesion derived on the basis of results of five random sampling trials is shown with respect to sTPD, dTPD, TIDr, and the case of the diagnosis assistance device 100b.

The AUC was calculated with respect to sTPD, dTPD, TIDr, and the case of the diagnosis assistance device 100b of the embodiment. The AUC for sTPD was 0.80, the AUC for dTPD was 0.83, the AUC for TIDr was 0.49, and the AUC for the case of the diagnosis assistance device 100b of the embodiment was 0.89. It was shown that the diagnosis assistance device 100b of the present embodiment has significantly improved diagnostic capability compared to the conventional TIDr. Similar results were obtained with the diagnosis assistance device 100a.

FIG. 25B shows results of comparing sensitivity, specificity, and accuracy with respect to sTPD, dTID, TIDr, and the case of the diagnosis assistance device 100b of the embodiment. In FIG. 25B, the vertical axis represents a proportion where P<0.05. P indicates a significant difference. According to FIG. 25B, it can be seen that the diagnosis assistance device 100b of the present embodiment has improved not only the sensitivity but also the specificity and accuracy.

### CONFIGURATION EXAMPLES

As an example configuration, there is provided a diagnosis assistance device including: a reception unit configured to receive an automatically quantified value of myocardial ischemia of a subject and cardiac function information and phase information acquired when stress myocardial scintigraphy is performed on the subject; a processing unit including a trained model for acquiring at least one of a predicted value of a result of performing reperfusion therapy, a result of predicting the onset of heart failure, a result of predicting cardiac death, a result of predicting all-cause mortality, and a result of predicting coronary artery disease on the basis of the automatically quantified value of the myocardial ischemia, the cardiac function information, and the phase information received by the reception unit and a trained model; and an output unit configured to output at least one of the predicted value of the result of performing the reperfusion therapy, the result of predicting the onset of the heart failure, the result of predicting the cardiac death, the result of predicting the all-cause mortality, and the result of predicting the coronary artery disease acquired by the processing unit, wherein the phase information is obtained by acquiring a phase of an increase or a decrease in a gamma ray count for contraction and expansion of a heart in each part of myocardium in a region of interest on an image associated with the contraction and expansion of the heart according to video information of the stress myocardial scintigraphy, and wherein the trained model is obtained by performing machine learning on a relationship between a combination of the automatically quantified value of the myocardial ischemia, the cardiac function information, and the phase information and at least one of the predicted value of the result of performing the reperfusion therapy, the result of predicting the onset of the heart failure, the result of predicting the cardiac death, the result of predicting the all-cause mortality, and the result of predicting the coronary artery disease.

As a configuration example, the reception unit receives a visual semi-quantitative index of the subject and the cardiac function information and the phase information acquired when the stress myocardial scintigraphy is performed on the subject, the processing unit acquires at least one of the predicted value of the result of performing the reperfusion therapy, the result of predicting the onset of the heart failure, the result of predicting the cardiac death, the result of predicting the all-cause mortality, and the result of predicting the coronary artery disease on the basis of the visual semi-quantitative index, the cardiac function information, and the phase information received by the reception unit, and the trained model is obtained by performing machine learning on a relationship between a combination of the visual semi-quantitative index, the cardiac function information, and the phase information and at least one of the predicted value of the result of performing the reperfusion therapy, the result of predicting the onset of the heart failure, the result of predicting the cardiac death, the result of predicting the all-cause mortality, and the result of predicting the coronary artery disease.

As a configuration example, the reception unit receives at least one of an index for predicting the onset of coronary artery disease acquired before the stress myocardial scintigraphy is performed on the subject, a coronary artery calcium score of the subject acquired when the stress myocardial scintigraphy is performed, a body mass index of the subject, and a left ventricular volume ratio during stress and rest of the subject, the processing unit acquires at least one of the predicted value of the result of performing the reperfusion therapy, the result of predicting the onset of the heart failure, the result of predicting the cardiac death, the result of predicting the all-cause mortality, and the result of predicting the coronary artery disease on the basis of at least one of the index for predicting the onset of the coronary artery disease, the coronary artery calcium score, the body mass index, and the left ventricular volume ratio received by the reception unit, the automatically quantified value of the myocardial ischemia, the cardiac function information, the phase information, and the trained model, and the trained model is obtained by performing machine learning on a relationship between a combination of at least one of the index for predicting the onset of the coronary artery disease, the coronary artery calcium score, the body mass index, and the left ventricular volume ratio, the automatically quantified value of the myocardial ischemia, the cardiac function information, and the phase information and at least one of the predicted value of the result of performing the reperfusion therapy, the result of predicting the onset of the heart failure, the result of predicting the cardiac death, the result of predicting the all-cause mortality, and the result of predicting the coronary artery disease.

As a configuration example, the cardiac function information includes a left ventricular ejection fraction.

As a configuration example, the phase information includes one or both of a phase bandwidth and an entropy.

As a configuration example, there is provided a learning model creation device including: a reception unit configured to receive a learning dataset in which an automatically quantified value of myocardial ischemia of a subject and cardiac function information and phase information acquired when stress myocardial scintigraphy is performed on the subject is included as learning data and at least one of a predicted value of a result of performing reperfusion therapy on the subject, a result of the onset of heart failure, a result of cardiac death, a result of all-cause mortality, and a result of coronary artery disease is included as training data; a processing unit configured to create a learning model by performing machine learning on a relationship of the automatically quantified value of the myocardial ischemia, the cardiac function information, the phase information, and at least one of the predicted value of the result of performing the reperfusion therapy, the result of the onset of the heart failure, the result of the cardiac death, the result of the all-cause mortality, and the result of the coronary artery disease using the automatically quantified value of the myocardial ischemia, the cardiac function information, and the phase information as explanatory variables and using at least one of the predicted value of the result of performing the reperfusion therapy, the result of the onset of the heart failure, the result of the cardiac death, the result of the all-cause mortality, and the result of the coronary artery disease as an objective variable on the basis of the learning dataset received by the reception unit; and an output unit configured to output the learning model created by the processing unit, wherein the phase information is obtained by acquiring a phase of an increase or a decrease in a gamma ray count in each part of myocardium in a region of interest on an image associated with contraction and expansion of a heart according to video information of the stress myocardial scintigraphy.

As a configuration example, the reception unit receives a learning dataset in which a visual semi-quantitative index of the subject and the cardiac function information and the phase information acquired when the stress myocardial scintigraphy is performed on the subject is included as learning data and at least one of the predicted value of the result of performing the reperfusion therapy on the subject, the result of the onset of the heart failure, the result of the cardiac death, the result of the all-cause mortality, and the result of the coronary artery disease is included as training data, and the processing unit configured to create a learning model by performing machine learning on a relationship of the visual semi-quantitative index, the cardiac function information, the phase information, and at least one of the predicted value of the result of performing the reperfusion therapy, the result of the onset of the heart failure, the result of the cardiac death, the result of the all-cause mortality, and the result of the coronary artery disease using the visual semi-quantitative index, the cardiac function information, and the phase information as explanatory variables and using at least one of the predicted value of the result of performing the reperfusion therapy, the result of the onset of the heart failure, the result of the cardiac death, the result of the all-cause mortality, and the result of the coronary artery disease as an objective variable on the basis of the learning dataset received by the reception unit.

As a configuration example, the reception unit receives a learning dataset in which at least one of an index for predicting the onset of coronary artery disease acquired before the stress myocardial scintigraphy is performed on the subject, a coronary artery calcium score of the subject acquired when the stress myocardial scintigraphy is performed, a body mass index of the subject, and a left ventricular volume ratio during stress and rest of the subject is further included as learning data, and the processing unit creates a learning model by performing machine learning on a relationship of the automatically quantified value of the myocardial ischemia, the cardiac function information, the phase information, and at least one of the predicted value of the result of performing the reperfusion therapy, the result of the onset of the heart failure, the result of the cardiac death, the result of the all-cause mortality, and the result of the coronary artery disease using at least one of the index for predicting the onset of the coronary artery disease, the coronary artery calcium score, the body mass index, and the left ventricular volume ratio, the automatically quantified value of the myocardial ischemia, the cardiac function information, and the phase information as explanatory variables and using at least one of the predicted value of the result of performing the reperfusion therapy, the result of the onset of the heart failure, the result of the cardiac death, the result of the all-cause mortality, and the result of the coronary artery disease as an objective variable on the basis of the learning dataset received by the reception unit.

Although the embodiments and the modified examples of the embodiments of the present invention have been described in detail above with reference to the drawings, the specific configurations are not limited to these embodiments and the modified examples of the embodiments, and design changes and the like are included without departing the scope and spirit of the present invention. For example, Modified Example 1 of the embodiment and Modified Example 2 of the embodiment may be combined.

Also, a computer program for implementing the functions of the diagnosis assistance device 100, the diagnosis assistance device 100a, the diagnosis assistance device 100b, the learning model creation device 200, the learning model creation device 200a, and the learning model creation device 200b described above may be recorded on a computer-readable recording medium and the program stored in the recording medium may be read and executed by a computer system. Also, the “computer system” used here may include an operating system (OS) or hardware such as peripheral devices.

Also, the “computer-readable recording medium” refers to a flexible disk, a magneto-optical disc, a read-only memory (ROM), a writable nonvolatile memory such as a flash memory, a portable medium such as a digital versatile disc (DVD), or a storage device such as a hard disk embedded in the computer system.

Furthermore, the “computer-readable recording medium” is assumed to include a computer-readable recording medium for retaining the program for a predetermined time period as in a volatile memory (for example, a dynamic random access memory (DRAM)) inside the computer system including a server and a client when the program is transmitted via a network such as the Internet or a communication circuit such as a telephone circuit.

Moreover, the above-described program may be transmitted from a computer system storing the program in a storage device or the like via a transmission medium or transmitted to another computer system by transmission waves in a transmission medium. Here, the “transmission medium” for transmitting the program refers to a medium having a function of transmitting information as in a network (a communication network) such as the Internet or a communication circuit (a communication line) such as a telephone circuit.

Moreover, the above-described program may be a program for implementing some of the above-described functions. Further, the above-described program may be a program capable of implementing the above-described function in combination with a program already recorded on the computer system. i.e., a so-called differential file (differential program).

### REFERENCE SIGNS LIST

100, 100a, 100b Diagnosis assistance device
102 Input unit
104 Reception unit
106, 106a, 106b Processing unit
107, 107a, 107b Trained model
108 Output unit
110 Storage unit
200, 200a, 200b Learning model creation device
202 Input unit
204 Reception unit
206, 206a, 206b Processing unit
207, 207a, 207b, Learning model
208 Output unit
210 Storage unit

1. A diagnosis assistance device comprising:
a reception unit configured to receive an automatically quantified value of myocardial ischemia of a subject and cardiac function information and phase information acquired when stress myocardial scintigraphy is performed on the subject;
a processing unit including a trained model for acquiring at least one of a predicted value of a result of performing reperfusion therapy, a result of predicting the onset of heart failure, a result of predicting cardiac death, a result of predicting all-cause mortality, and a result of predicting coronary artery disease on the basis of the automatically quantified value of the myocardial ischemia, the cardiac function information, and the phase information received by the reception unit and a trained model; and
an output unit configured to output at least one of the predicted value of the result of performing the reperfusion therapy, the result of predicting the onset of the heart failure, the result of predicting the cardiac death, the result of predicting the all-cause mortality, and the result of predicting the coronary artery disease acquired by the processing unit,
wherein the phase information is obtained by acquiring a phase of an increase or a decrease in a gamma ray count for contraction and expansion of a heart in each part of myocardium in a region of interest on an image associated with the contraction and expansion of the heart according to video information of the stress myocardial scintigraphy, and
wherein the trained model is obtained by performing machine learning on a relationship between a combination of the automatically quantified value of the myocardial ischemia, the cardiac function information, and the phase information and at least one of the predicted value of the result of performing the reperfusion therapy, the result of predicting the onset of the heart failure, the result of predicting the cardiac death, the result of predicting the all-cause mortality, and the result of predicting the coronary artery disease.

2. The diagnosis assistance device according to claim 1,
wherein the reception unit receives a visual semi-quantitative index of the subject and the cardiac function information and the phase information acquired when the stress myocardial scintigraphy is performed on the subject,
wherein the processing unit acquires at least one of the predicted value of the result of performing the reperfusion therapy, the result of predicting the onset of the heart failure, the result of predicting the cardiac death, the result of predicting the all-cause mortality, and the result of predicting the coronary artery disease on the basis of the visual semi-quantitative index, the cardiac function information, and the phase information received by the reception unit, and
wherein the trained model is obtained by performing machine learning on a relationship between a combination of the visual semi-quantitative index, the cardiac function information, and the phase information and at least one of the predicted value of the result of performing the reperfusion therapy, the result of predicting the onset of the heart failure, the result of predicting the cardiac death, the result of predicting the all-cause mortality, and the result of predicting the coronary artery disease.

3. The diagnosis assistance device according to claim 1 or 2,
wherein the reception unit receives at least one of an index for predicting the onset of coronary artery disease acquired before the stress myocardial scintigraphy is performed on the subject, a coronary artery calcium score of the subject acquired when the stress myocardial scintigraphy is performed, a body mass index of the subject, and a left ventricular volume ratio during stress and rest of the subject,
wherein the processing unit acquires at least one of the predicted value of the result of performing the reperfusion therapy, the result of predicting the onset of the heart failure, the result of predicting the cardiac death, the result of predicting the all-cause mortality, and the result of predicting the coronary artery disease on the basis of at least one of the index for predicting the onset of the coronary artery disease, the coronary artery calcium score, the body mass index, and the left ventricular volume ratio received by the reception unit, the automatically quantified value of the myocardial ischemia, the cardiac function information, the phase information, and the trained model, and
wherein the trained model is obtained by performing machine learning on a relationship between a combination of at least one of the index for predicting the onset of the coronary artery disease, the coronary artery calcium score, the body mass index, and the left ventricular volume ratio, the automatically quantified value of the myocardial ischemia, the cardiac function information, and the phase information and at least one of the predicted value of the result of performing the reperfusion therapy, the result of predicting the onset of the heart failure, the result of predicting the cardiac death, the result of predicting the all-cause mortality, and the result of predicting the coronary artery disease.

4. The diagnosis assistance device according to claim 1, wherein the cardiac function information includes a left ventricular ejection fraction.

5. The diagnosis assistance device according to claim 1, wherein the phase information includes at least one of a standard deviation, a phase bandwidth, and an entropy obtained from measurement of timings of contraction and expansion of the myocardium.

6. A learning model creation device comprising:
a reception unit configured to receive a learning dataset in which an automatically quantified value of myocardial ischemia of a subject and cardiac function information and phase information acquired when stress myocardial scintigraphy is performed on the subject is included as learning data and at least one of a predicted value of a result of performing reperfusion therapy on the subject, a result of the onset of heart failure, a result of cardiac death, a result of all-cause mortality, and a result of coronary artery disease is included as training data;
a processing unit configured to create a learning model by performing machine learning on a relationship of the automatically quantified value of the myocardial ischemia, the cardiac function information, the phase information, and at least one of the predicted value of the result of performing the reperfusion therapy, the result of the onset of the heart failure, the result of the cardiac death, the result of the all-cause mortality, and the result of the coronary artery disease using the automatically quantified value of the myocardial ischemia, the cardiac function information, and the phase information as explanatory variables and using at least one of the predicted value of the result of performing the reperfusion therapy, the result of the onset of the heart failure, the result of the cardiac death, the result of the all-cause mortality, and the result of the coronary artery disease as an objective variable on the basis of the learning dataset received by the reception unit; and
an output unit configured to output the learning model created by the processing unit,
wherein the phase information is obtained by acquiring a phase of an increase or a decrease in a gamma ray count in each part of myocardium in a region of interest on an image associated with contraction and expansion of a heart according to video information of the stress myocardial scintigraphy.

7. The learning model creation device according to claim 6,
wherein the reception unit receives a learning dataset in which a visual semi-quantitative index of the subject and the cardiac function information and the phase information acquired when the stress myocardial scintigraphy is performed on the subject is included as learning data and at least one of the predicted value of the result of performing the reperfusion therapy on the subject, the result of the onset of the heart failure, the result of the cardiac death, the result of the all-cause mortality, and the result of the coronary artery disease is included as training data, and
wherein the processing unit configured to create a learning model by performing machine learning on a relationship of the visual semi-quantitative index, the cardiac function information, the phase information, and at least one of the predicted value of the result of performing the reperfusion therapy, the result of the onset of the heart failure, the result of the cardiac death, the result of the all-cause mortality, and the result of the coronary artery disease using the visual semi-quantitative index, the cardiac function information, and the phase information as explanatory variables and using at least one of the predicted value of the result of performing the reperfusion therapy, the result of the onset of the heart failure, the result of the cardiac death, the result of the all-cause mortality, and the result of the coronary artery disease as an objective variable on the basis of the learning dataset received by the reception unit.

8. The learning model creation device according to claim 6,
wherein the reception unit receives a learning dataset in which at least one of an index for predicting the onset of coronary artery disease acquired before the stress myocardial scintigraphy is performed on the subject, a coronary artery calcium score of the subject acquired when the stress myocardial scintigraphy is performed, a body mass index of the subject, and a left ventricular volume ratio during stress and rest of the subject is further included as learning data, and
wherein the processing unit creates a learning model by performing machine learning on a relationship of the automatically quantified value of the myocardial ischemia, the cardiac function information, the phase information, and at least one of the predicted value of the result of performing the reperfusion therapy, the result of the onset of the heart failure, the result of the cardiac death, the result of the all-cause mortality, and the result of the coronary artery disease using at least one of the index for predicting the onset of the coronary artery disease, the coronary artery calcium score, the body mass index, and the left ventricular volume ratio, the automatically quantified value of the myocardial ischemia, the cardiac function information, and the phase information as explanatory variables and using at least one of the predicted value of the result of performing the reperfusion therapy, the result of the onset of the heart failure, the result of the cardiac death, the result of the all-cause mortality, and the result of the coronary artery disease as an objective variable on the basis of the learning dataset received by the reception unit.

9. A diagnosis assistance method to be executed by a computer, the diagnosis assistance method comprising:
a step of receiving an automatically quantified value of myocardial ischemia of a subject and cardiac function information and phase information acquired when stress myocardial scintigraphy is performed on the subject;
a step of acquiring at least one of a predicted value of a result of performing reperfusion therapy, a result of predicting the onset of heart failure, a result of predicting cardiac death, a result of predicting all-cause mortality, and a result of predicting coronary artery disease on the basis of the automatically quantified value of the myocardial ischemia, the cardiac function information, and the phase information received in the receiving step and a trained model; and
a step of outputting at least one of the predicted value of the result of performing the reperfusion therapy, the result of predicting the onset of the heart failure, the result of predicting the cardiac death, the result of predicting the all-cause mortality, and a predicted value of the result of predicting the coronary artery disease that have been acquired,
wherein the phase information is obtained by acquiring a phase of an increase or a decrease in a gamma ray count in each part of myocardium in a region of interest on an image associated with contraction and expansion of a heart according to video information of the stress myocardial scintigraphy, and
wherein the trained model is obtained by performing machine learning on a relationship between a combination of the automatically quantified value of the myocardial ischemia, the cardiac function information, and the phase information and at least one of the predicted value of the result of performing the reperfusion therapy, the result of predicting the onset of the heart failure, the result of predicting the cardiac death, the result of predicting the all-cause mortality, and the result of predicting the coronary artery disease.

10. A learning model creation method to be executed by a computer, the learning model creation method comprising:
a step of receiving a learning dataset in which an automatically quantified value of myocardial ischemia of a subject and cardiac function information and phase information acquired when stress myocardial scintigraphy is performed on the subject is included as learning data and at least one of a predicted value of a result of performing reperfusion therapy on the subject, a result of the onset of heart failure, a result of cardiac death, a result of all-cause mortality, and a result of coronary artery disease is included as training data;
a step of creating a learning model by performing machine learning on a relationship of the automatically quantified value of the myocardial ischemia, the cardiac function information, the phase information, and at least one of the predicted value of the result of performing the reperfusion therapy, the result of the onset of the heart failure, the result of the cardiac death, the result of the all-cause mortality, and the result of the coronary artery disease using the automatically quantified value of the myocardial ischemia, the cardiac function information, and the phase information as explanatory variables and using at least one of the predicted value of the result of performing the reperfusion therapy, the result of the onset of the heart failure, the result of the cardiac death, the result of the all-cause mortality, and the result of the coronary artery disease as an objective variable on the basis of the learning dataset received in the receiving step; and
a step of outputting the learning model created in the creating step,
wherein the phase information is obtained by acquiring a phase of an increase or a decrease in a gamma ray count in each part of myocardium in a region of interest on an image associated with contraction and expansion of a heart according to video information of the stress myocardial scintigraphy.

11. A program for causing a computer to execute:
a step of receiving an automatically quantified value of myocardial ischemia of a subject and cardiac function information and phase information acquired when stress myocardial scintigraphy is performed on the subject;
a step of acquiring at least one of a predicted value of a result of performing reperfusion therapy, a result of predicting the onset of heart failure, a result of predicting cardiac death, a result of predicting all-cause mortality, and a result of predicting coronary artery disease on the basis of the automatically quantified value of the myocardial ischemia, the cardiac function information, and the phase information received in the receiving step and a trained model; and
a step of outputting at least one of the predicted value of the result of performing the reperfusion therapy, the result of predicting the onset of the heart failure, the result of predicting the cardiac death, the result of predicting the all-cause mortality, and the result of predicting the coronary artery disease that have been acquired,
wherein the phase information is obtained by acquiring a phase of an increase or a decrease in a gamma ray count in each part of myocardium in a region of interest on an image associated with contraction and expansion of a heart according to video information of the stress myocardial scintigraphy, and
wherein the trained model is obtained by performing machine learning on a relationship between a combination of the automatically quantified value of the myocardial ischemia, the cardiac function information, and the phase information and at least one of the predicted value of the result of performing the reperfusion therapy, the result of predicting the onset of the heart failure, the result of predicting the cardiac death, the result of predicting the all-cause mortality, and the result of predicting the coronary artery disease.

12. A program for causing a computer to execute:
a step of receiving a learning dataset in which an automatically quantified value of myocardial ischemia of a subject and cardiac function information and phase information acquired when stress myocardial scintigraphy is performed on the subject is included as learning data and at least one of a predicted value of a result of performing reperfusion therapy on the subject, a result of the onset of heart failure, a result of cardiac death, a result of all-cause mortality, and a result of coronary artery disease is included as training data;
a step of creating a learning model by performing machine learning on a relationship of the automatically quantified value of the myocardial ischemia, the cardiac function information, the phase information, and at least one of the predicted value of the result of performing the reperfusion therapy, the result of the onset of the heart failure, the result of the cardiac death, the result of the all-cause mortality, and the result of the coronary artery disease using the automatically quantified value of the myocardial ischemia, the cardiac function information, and the phase information as explanatory variables and using at least one of the predicted value of the result of performing the reperfusion therapy, the result of the onset of the heart failure, the result of the cardiac death, the result of the all-cause mortality, and the result of the coronary artery disease as an objective variable on the basis of the learning dataset received in the receiving step; and
a step of outputting the learning model created in the creating step,
wherein the phase information is obtained by acquiring a phase of an increase or a decrease in a gamma ray count in each part of myocardium in a region of interest on an image associated with contraction and expansion of a heart according to video information of the stress myocardial scintigraphy.
